(19) United States
(12) Patent Application Publication
(10) Pub. No.: US 2016/0136385 A1
(43) Pub. Date: May 19, 2016

(54) SYSTEM AND METHOD FOR THERMALLY CONDITIONING A SLEEP ENVIRONMENT AND MANAGING SKIN TEMPERATURE OF A USER

(71) Applicant: Ruggero Scorcioni, San Diego, CA (US)

(72) Inventor: Ruggero Scorcioni, San Diego, CA (US)

(21) Appl. No.: 14/940,509

(22) Filed: Nov. 13, 2015

Related U.S. Application Data
(60) Provisional application No. 62/080,140, filed on Nov. 14, 2014.

(51) Int. Cl. A61M 21/02; A47C 21/04

(52) U.S. Cl. CPC A61M 21/02; A47C 21/048; A47C 21/042; A61M 2021/0066

(57) ABSTRACT

A system and method is provided for thermally conditioning a sleep environment and managing skin temperature of a user. The thermal system and method include a heat exchanger and controller configured to conductively cool and heat a user during sleep and at other times. The thermal system provides for management of skin temperature of a user, such that sleep comfort and/or quality may be improved, and the thermal system may take advantage of reduced sensitivity of a sleeper during deep sleep for more aggressive thermal manipulation of skin temperature via the conductive cooling. In addition, the thermal system may be further configured to condition the sleep environment when the user is not present based on pre-sleep or maintenance considerations.

### TECHNICAL FIELD

The present disclosure generally pertains to a source of, or a sink for, thermal energy so associated with a sleeping environment, such as a bed, as to affect the temperature felt by a person in the sleeping environment, and is more particularly directed toward a thermal system for conditioning the sleep environment and managing skin temperature of a user during sleep.

### BACKGROUND OF THE INVENTION

Sleep is essential for a person's health and wellbeing, yet millions of people do not get enough sleep and many suffer from lack of sleep. Surveys conducted by the U.S. National Science Foundation between 1999 and 2004 found that at least 40 million Americans suffer from over 70 different sleep disorders, and 60 percent of adults report having sleep problems a few nights a week or more. Most of those with these problems go undiagnosed and untreated.

Disruptions in sleep can be caused by a variety of issues, from teeth grinding (bruxism) to uncomfortable sleep environment to night terrors. Some common sleep disorders also include sleep apnea (stops in breathing during sleep), narcolepsy and hypersomnia (excessive sleepiness at inappropriate times), cataplexy (sudden and transient loss of muscle tone while awake), and sleeping sickness (disruption of sleep cycle due to infection). When a person suffers from difficulty falling asleep and/or staying asleep with no obvious cause, it is referred to as insomnia. An uncomfortable sleep environment may be disruptive to sleep and may contribute or aggravate another sleep condition. An uncomfortable sleep environment may be due to uncomfortable bedding, noise, excessively high or low temperatures, light, etc.

U.S. Pat. No. 5,448,788, issued to Wu, shows a thermoelectric cooling-heating mattress. In particular, the thermostat controlled mattress includes a mattress unit having an underlay, a surface cover and a curved circuit. A water circuit tube connects to the curved circuit so as to allow water to be introduced into the mattress unit with the aid of a pump. Water is circulated between the mattress unit and a water storage box via the water circuit tube. A sensor is operatively arranged with respect to the water storage box to sense the temperature and quantity of water contained in the water storage box and sends a signal to a thermostat electric circuit. An aluminum reservoir for the water is connected to the curved circuit of the mattress unit and the water circuit tube. A thermoelectric element is connected to the reservoir and the power supply to heat or cool the water. Water is circulated in the water circuit tube between the curved circuit of the mattress unit and the water storage box, through the reservoir. The water temperature is controlled based on signals generated by the thermostat electric circuit, which activates the power supply operatively connected to the thermoelectric element. A heat sink and a fan may be arranged adjacent to the thermoelectric element such that the fan blows a current of air onto the heat sink.

U.S. Pat. No. 8,146,833, issued to Song et al., shows a method to control sleep operation of air conditioner. In particular, the method includes determining whether or not a sleep operation is activated and sequentially performing a plurality of sub-modes of the sleep operation when the sleep operation is activated. An aspect of the method is to control a sleep operation of an air conditioner, wherein an indoor temperature is automatically changed in the sleep operation.

U.S. Pat. No. 8,690,751, issued to Auphan, shows a sleep and environment control method and system. In particular, the sleep system is provided that aids in achieving a sleep goal by controlling the environment near a person. The sleep system executes instructions on a processor that interfaces with the person and various environmental controls. As the instructions are executed, the sleep system receives a sleep goal for the person that includes varying the nearby environment. The processor may further execute instructions to create settings that vary at least one environmental condition of the environment over time as it relates to one or more cycles of a sleep architecture for the person. Varying at least one environmental condition near the person experiencing one or more cycles of the sleep architecture influences the quality of the person's sleep. The sleep system may further adjust at least one environmental condition in the vicinity of the person tailored to the sleep architecture for the person. Auphan fails to teach which specific types of changes are required to achieve better sleep, however.

Previous disclosures describe maintaining a comfortable temperature while sleeping, lightly varying a sleep environment temperature during sleep, controlling the temperature of an entire room, or providing convective temperature control that may actually aggravate certain types of sleep disorders. The present disclosure is directed toward overcoming known problems as well as additional problems discovered by the inventor.

### BRIEF SUMMARY OF THE INVENTION

A system for thermally conditioning a sleep environment is disclosed herein. The system for thermally conditioning a sleep environment includes a heat exchanger configured to conductively heat and cool a user, a controller configured to operate the heat exchanger according to a sleeping mode and a waking mode, the sleeping mode occurring between a begin-sleeping time and a begin-waking time, the sleeping mode including conductively cooling the user along a thermal-comfort profile to a minimum temperature, the thermal-comfort profile and the minimum temperature above a threshold determined as disruptive to sleeping, the waking mode occurring between the begin-waking time and an waking time, the waking mode including conductively warming the user toward a waking temperature at the waking time.

According to one embodiment a system for managing skin temperature of a user is disclosed herein. The system for managing skin temperature of a user includes a user interface heat exchanger configured to conductively heat and cool a user and a heat pump module fluidly coupled to the user interface heat exchanger, the heat pump module configured to conductively heat and conductively cool the user. The system for managing skin temperature of a user further includes a controller communicably coupled to the heat pump module, the controller configured to operate at least one of the heat pump module according to a sleeping mode and a warming mode, the sleeping mode occurring between a begin-sleeping time and a begin-warming time, the sleeping mode including conductively cooling the user along a thermal-comfort profile to a minimum temperature, the thermal-comfort profile and the minimum temperature being proximate and above a threshold determined as disruptive to sleeping, and the warming mode occurring between the begin-warming time and a waking time, the warming mode including conductively warming the user toward a waking temperature at the waking time.

According to another embodiment, a method for thermally conditioning a sleep environment is also disclosed herein. The method for thermally conditioning a sleep environment providing a thermal system including a heat exchanger, the heat exchanger configured to conductively heat and cool a user, operating a sleeping mode of the thermal system, including conductively cooling the user along a thermal-comfort profile to a minimum temperature between a begin-sleeping time and a begin-warming time, the thermal-comfort profile and the minimum temperature being above a threshold thermal profile determined as disruptive to sleeping, and operating a warming mode of the thermal system, including conductively warming the user toward a waking temperature between the begin-warming time and a waking time.

### BRIEF DESCRIPTION OF THE DRAWINGS

FIG. 1 schematically illustrates a thermal system for conditioning a sleep environment.

FIG. 2 schematically illustrates an exemplary controller of the thermal system of FIG. 1.

FIG. 3 schematically illustrates an exemplary heat exchanger of the thermal system of FIG. 1.

FIG. 4A illustrates a front view of one exemplary embodiment of the heat pump module of FIG. 3.

FIG. 4B illustrates a side view of one exemplary embodiment of the heat pump module of FIG. 3.

FIG. 4C illustrates a back view of one exemplary embodiment of the heat pump module of FIG. 3.

FIG. 4D illustrates a front view of another exemplary embodiment of the heat pump module of FIG. 3.

FIG. 5A schematically illustrates an exemplary sensor array configuration of the thermal system of FIG. 1.

FIG. 5B schematically illustrates an exemplary sensor unit of the thermal system of FIG. 1.

FIG. 5C schematically illustrates another exemplary sensor configuration including sensor strips of the thermal system of FIG. 1.

FIG. 6A illustrates exemplary thermal-comfort profiles and exemplary operation modes of the controller of the thermal system of FIG. 1.

FIG. 6B illustrates an exemplary thermal-comfort profile and exemplary operation modes of the controller of the thermal system of FIG. 1.

FIG. 6C illustrates an exemplary thermal-comfort profile and exemplary operation modes of the controller of the thermal system of FIG. 1.

FIG. 6D illustrates exemplary thermal-comfort profiles and exemplary operation modes of the controller of the thermal system of FIG. 1.

FIG. 7 is a flow chart of an exemplary method for conditioning a sleep environment.

### DETAILED DESCRIPTION

Various aspects of the novel systems, apparatuses, and methods are described more fully hereinafter with reference to the accompanying drawings. The detailed description set forth below in connection with the appended drawings is intended as a description of various configurations and is not intended to represent the only configurations in which the concepts described herein may be practiced. The detailed description includes specific details for the purpose of providing a thorough understanding of various concepts. In particular, aspects of the present disclosure relate to a system and method for thermally conditioning a sleep environment and managing skin temperature of a user during sleep. Embodiments of the system and method are directed to both heating and cooling the user dynamically upon the detection of various states and preferences of the user throughout the use of a bed, for example. However, it will be apparent to those skilled in the art that these concepts may be practiced without these specific details. In some instances, well known structures and components are shown in block diagram form in order to avoid obscuring such concepts.

In the following description, for purposes of explanation, numerous specific details are set forth in order to provide a thorough understanding of one or more aspects. It may be evident, however, that such aspect(s) may be practiced without these specific details. For example, although reference is made to a sleep environment, the present disclosure may relate more broadly to many environments including, but not limited to, a rest environment, a therapeutic environment, an entertainment environment, a performance enhancing environment, etc. Also for example, although reference is made to a bed, the present disclosure may relate to many devices intended to receive the human body in a prone, supine, or sitting position for the purpose of repose, examination, or treatment. Thus, in addition to beds, devices ordinarily known as examining tables, operating tables, hammocks, cradles, cribs, cots, camp beds, ground mats, sleeping bags, and bed accessories, such as mattresses, pillows, surgical supports, and bed clothing may be included. Furthermore, the system may incorporate portions of each, such as a pad or mat placed upon the bed (or the like), a cover placed above a user on the bed (or the like), or a combination thereof.

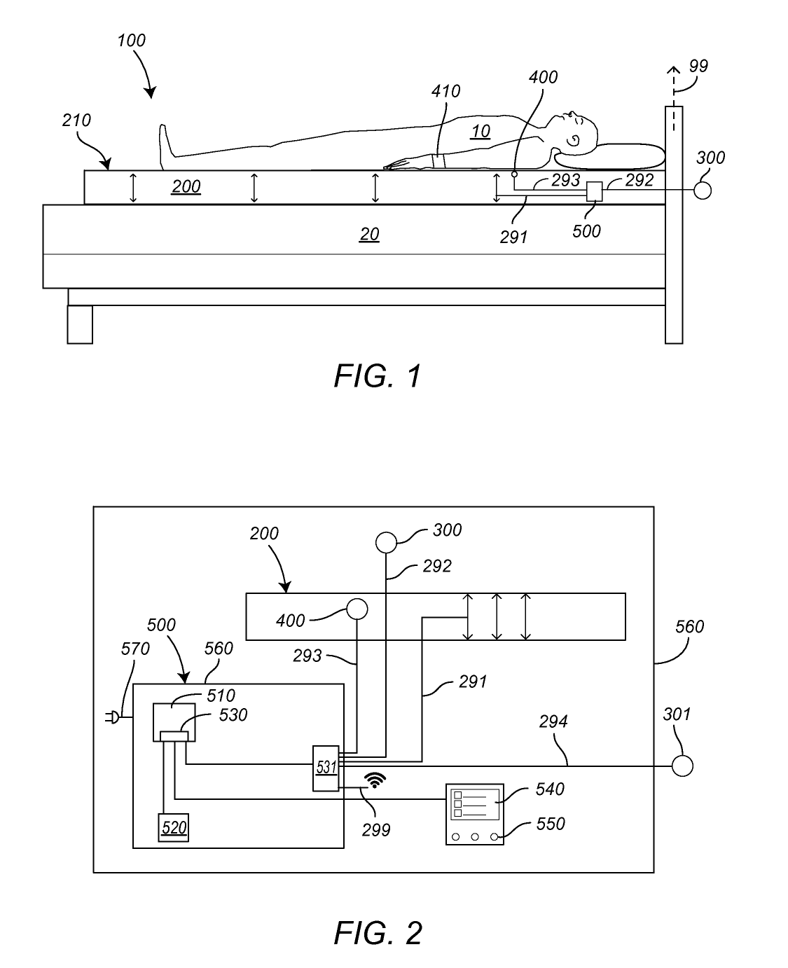

FIG. 1 schematically illustrates a system for thermally conditioning a sleep environment (hereinafter “thermal system”). In particular, thermal system 100 is shown according to one exemplary embodiment, including a heat exchanger 200 and a controller 500. Here and in other figures, reference may be made to a “top” side, direction, or surface; “top” is conveniently defined to correspond to an uppermost elevation when the thermal system 100 is deployed. For clarity, this should be commonly be understood in sense of “the top of a bed”, and is indicated by elevation axis 99.

As illustrated, the heat exchanger 200 may arranged to set on top of a bed 20, such that a user 10 lays on top of the heat exchanger 200 when in bed. For example, the heat exchanger 200 (or a portion thereof) may include a generally planar portion extending across a sleeping area of the bed 20. Moreover, the heat exchanger 200 (or portion thereof) may unroll, unfold, or otherwise deploy, such as when integrated into a collapsible mat or pad. In other embodiments, the heat exchanger 200 may couple to, be incorporated into, or otherwise form part of the bed 20 (or other devices, as discussed above). Here, the heat exchanger 200 is represented as a single integrated unit, however, as discussed below, the heat exchanger 200 may be distributed into a plurality of sub units.

Generally, the controller 500 is configured to operate the heat exchanger 200 according to a sleeping mode and a waking mode, and the heat exchanger 200 is configured to conductively heat and cool (i.e., provide heat and remove heat from) the user 10, responsive to the controller 500. As discussed further below, the sleeping mode occurs between a begin-sleeping time and a begin-waking time, and is followed by the waking mode. The controller 500 may be further configured to operate the heat exchanger 200 according to a pre-sleeping mode, which precedes the sleeping mode. In addition, the controller 500 may be further configured to operate the heat exchanger 200 according to other modes, also discussed below.

According to one embodiment, conductively heating and cooling the user 10 may include the heat exchanger 200 raising and lowering the skin temperature of the user. In particular, the heat exchanger 200 may be configured to conductively heat and cool the user's skin via thermal conduction. For example, the heat exchanger 200 may include a user interface 210 having a thermally conductive top surface. As such, when the user 10 is lying on top of the user interface 210 of the heat exchanger 200, any portion of the user's skin in physical contact with the user interface 210 will likewise be in thermal contact with a portion of the thermally conductive surface. Alternately, the user interface 210 may include one or more thermally conductive circuits, elements, or areas, distributed across its top side, for example, so as to produce a similar net thermal effect or experience. For example, the user interface 210 may be configured as a solid state heat pump, such as a Peltier device configured to conductively heat the user 10 when powered in one polarity, and to conductively cool the user 10 when powered in the opposite polarity.

The heat exchanger 200 may be further controlled by the controller 500 such that the heating and cooling follows a thermal-comfort profile. As discussed below, the thermal-comfort profile may represent a temperature curve over time of a measured temperature (e.g., temperature of user interface 210), which is less than or just before reaching a threshold determined as disruptive to sleeping. The thermal-comfort profile may or may not significantly affect the user's core body temperature. However, embodiments may include configuring the thermal system to aggressively cool the user to just above the threshold defined or otherwise determined as being disruptive to the user's sleeping. For example, while aggressively cooling the user 10 along the thermal-comfort profile, the transmissible power to the user 10 may be at least 75 Watts of cooling (heat flow from the user). As such, the thermal system may be configured to provide said cooling.

Likewise, in some embodiments the thermal system may be configured to aggressively warm the user to just below a threshold defined or otherwise determined as being disruptive to the user's sleeping. For example, while aggressively warming the user 10 along the thermal-comfort profile, the transmissible power to the user 10 may be at least 75 Watts of heating (heat flow to the user). As such, the thermal system may be configured to provide said heating.

Also, the heating and cooling may be limited such that heat flow with the user 10 is limited to levels below that which may trigger closure of capillaries and/or change the user's core body temperature by greater than a nominal amount (e.g., +/−2 degree Celsius). For example, the heat flow through the user interface 210 and/or the thermal gradient between the user 10 and the user interface 210 may be restricted or otherwise controlled such that the user's 10 skin is in equilibrium, balanced by the user's 10 core body temperature.

According to one embodiment, the controller 500 may be configured to control heat flow with the user 10 based on the user's physical characteristics. In particular, the cooling and heating by the heat exchanger 200 may be further controlled based on the user's 10 weight or Body Mass Index (BMI). By adapting the thermal system to the physical characteristics of a particular user, the user's skin temperature may be managed at a more personal level without requiring the user to learn or manually adjust it. Moreover, the thermal system may be adapted to the physical characteristics of a particular user or a range of users, which may provide better sleep than a one-size-fits-all solution.

For example, according to one embodiment, the thermal system 100 may be configured to cool and/or heat the user 10 by at least 1 Watt per each kilogram of user weight. To illustrate, where user 10 weighs 80 Kg, the heat exchanger 200 may be configured to provide at least 80 Watts of cooling (i.e., heat removal) and/or 80 Watts of heating to the user 10. Accordingly, the heat exchanger 200 may include cooling and/or heating components with a rated maximum transmissible power of at least 80 Watts. Moreover, the heat exchanger 200 may be further adapted to accommodate a wide range of user weights and system efficiencies. For example, the heat exchanger 200 may include cooling and/or heating components with a rated maximum transmissible power of 150 Watts. According to another embodiment, the heat exchanger 200 may have or include cooling and/or heating components having a rated maximum transmissible power of 100 Watts to 300 Watts.

Also for example, according to another embodiment, the thermal system 100 may be configured to cool and/or heat the user 10 by at least three times the value of BMI of user 10, expressed in Watts. To illustrate, where the user 10 has a BMI of 30, the heat exchanger 200 may be configured to transmit at least 90 Watts of cooling and/or 90 Watts of heating to the user 10. As above, the heat exchanger 200 may be further adapted to accommodate a wide range of user BMIs and system efficiencies.

As described above, the heating and cooling power may include the power or heat removed from or provided to user 10 via user interface 210. The user 10 can be cooled or heated with other techniques that provide at least the same amount of cooling or heating power. For example user 10 can be cooled or heated conventionally via cold or hot gas, such as air blown through the mattress to the user 10. In another example user 10 can be radiantly cooled or radiantly heated by changing the temperature of sleep environment walls, or other surfaces not in contact with user 10. Here, the user 10 can either be uncovered, or covered by covers that do not shield thermal radiation.

According to one embodiment, the user's skin temperature can be cooled or heated by a combination of methods to improve sleep. In particular, the user 10 may be cooled using one technique or cooling mechanism (e.g., conduction, convection, radiation) and warmed using another technique or warming mechanism (e.g., conduction, convection, radiation). For example, the heat exchanger 200 may include a convection cooler and an electrical blanket or electrical pad, wherein the user 10 can be cooled by air blown under the blanket (i.e., between the mattress and bed cover or through the mattress) and warmed by the electrical blanket or electrical pad. Also for example, the heat exchanger 200 may include a conductive cooler and an electric blanket or electric pad, wherein the user 10 can be cooled by the heat exchanger 210 and heated by the electric blanket or electric pad.

According to one embodiment, the thermal system may be further configured to maintain air breathed by the user at a temperature independent of the cooling or heating of the user. In particular, the temperature of the air that user 10 breathes may be kept substantially constant while the heat exchanger 200 operates to cool and heat the user. Importantly, the thermal system may thermally condition the user's skin temperature, such as by conduction, while leaving the breathing air temperature practically unchanged (+/−2 degree Celsius) or actively maintaining the air temperature independently. For example, where the heat exchanger 200 operates by radiation, the temperature of the air breathed by the user may be kept constant (such as by a ducted air flow that is temperature controlled), while cooling and heating is applied by changing the temperature of surfaces not in contact with user 10. Beneficially, the user's skin may be managed for thermal comfort without substantially affecting his or her internal body temperature. Moreover, this may be very advantageous when more than one user sleeps in the same room, since each user may sleep with different timing, different sleep phase sequences, and may require different thermal skin profiles for optimal sleep, which need to be applied individually.

Advantageously, by configuring the user interface 210 for conductive heat exchange, the user may avoid sleep disruptions caused or triggered by convection cooling/heating. In particular, since conventional climate-controlled beds discharge cooled or heated air proximate the user (e.g., vented through a mattress), a user trying to sleep may be agitated by the stimulation of blowing air, or at least may need to develop a tolerance to it. This may be particularly the case where there is inadequate filtering of the convection media. Moreover, heated or cooled air reaching the nose and mouth may induce snoring, drying of the mouth, irritation of the throat, or even sleep apnea events. Furthermore, convection systems may cause further stimulation or sleep disruptions due to blower noise. Thus, as a conductive heat exchanger, the user interface 210 may beneficially provide a thermally comfortable sleep environment without the drawbacks of a conventional convection system and/or may extend the threshold determined as disruptive to sleeping, providing for a more “aggressive” thermal-comfort profile.

According to one embodiment, the thermal system 100 may further include an environment sensor 300 and/or a user sensor 400. The environment sensor 300 is configured to sense and communicate environmental conditions associated with the sleep environment. The user sensor 400 is configured to sense and communicate environmental conditions proximate the user 10.

Here, the environment sensor 300 and the user sensor 400 are distinguished from each other for clarity, and for the purpose of providing a thorough understanding of various concepts presented herein. However, it will be apparent to those skilled in the art that these concepts (as well as others throughout this disclosure) may be practiced without these specific details. In some instances, a single sensor may alternately function as the environment sensor 300 and the user sensor 400. Likewise, a single sensor may function as both the environment sensor 300 and the user sensor 400. In addition, the user sensor 400 or the environment sensor 300 may include or otherwise utilize a wearable user sensor 410.

While the heat exchanger 200 is conveniently diagrammed as a single unit for clarity, as discussed above, it may be distributed among a plurality of discrete units and/or combined with one or more other components of the thermal system 100. Likewise, as described further below, the environment sensor 300 and the user sensor 400 may each include a plurality of discrete sensors and associated componentry and/or be combined together or with one or more other components of the thermal system 100.

According to one embodiment, the heat exchanger 200 may be communicably coupled to the controller 500 via a first communication link 291. Similarly, the environment sensor 300 and the user sensor 400 may be communicably coupled to the controller 500 via a second and third communication link 292, 293, respectively. With each link, the controller 500 may communicate over any convenient media, such as a physical link or air link (e.g., over wire, wirelessly, or optically, to name a few). Furthermore, the communications on each communication link 291, 292, 293 may be uni- or bi-directional with the controller 500 other components of the thermal system 100. In addition, each communication link 291, 292, 293 may be independent from another, may be at least partially shared (e.g., over a system communication bus, a combined/shared communication link, a non-system communication link, etc.), or any combination thereof. Moreover, each illustrated communication link 291, 292, 293 may include a plurality of links, for example, where there are a plurality of components or subcomponents associated with each.

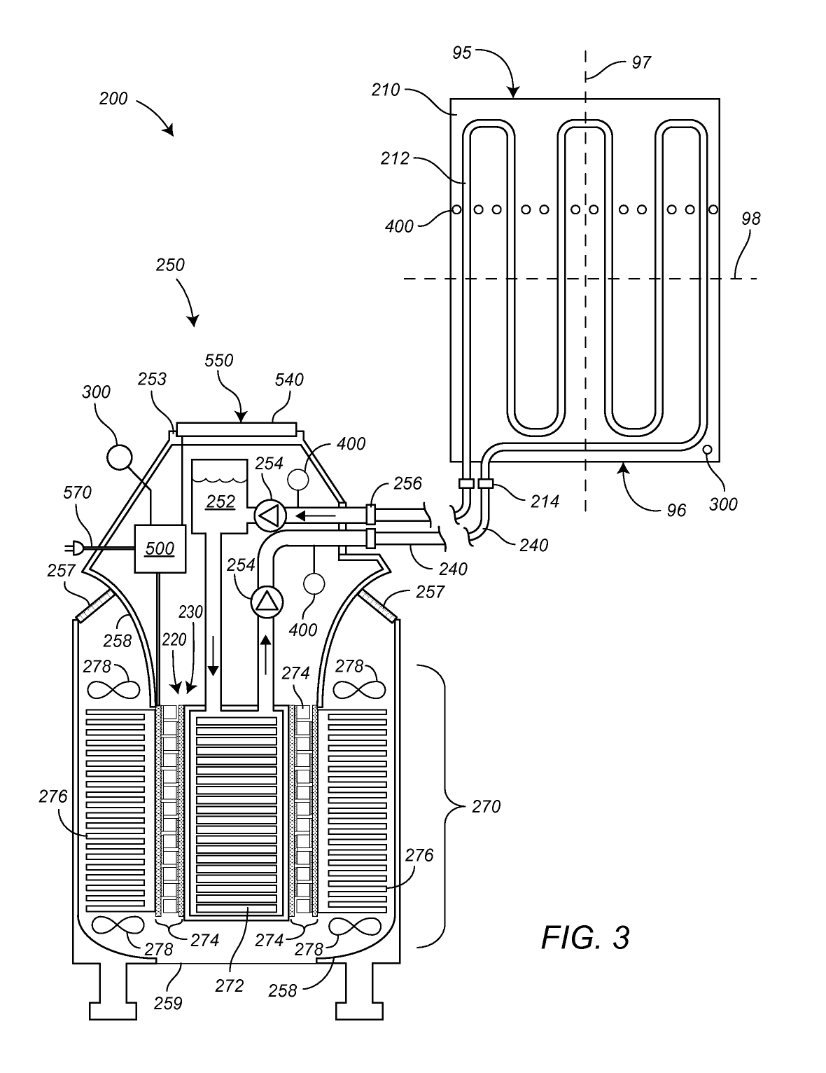

FIG. 2 schematically illustrates an exemplary controller of the thermal system of FIG. 1. The controller 500 may be implemented as a general purpose computer or a processing system. In particular, the controller 500 may include a processor and related features. Moreover, the processor and related features may include by various blocks, modules, components, circuits, steps, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system. For example, as illustrated, the controller 500 may include a processor 510, a memory 520, a communication port 530, a display 540, and a user control 550, which may be contained a housing 560 or on a chassis, and powered by a power supply 570.

As above, the controller 500 may be communicably coupled to the heat exchanger 200, the environment sensor 300, and the user sensor 400 via one or more communication links 291, 292, 293. The controller 500 may also be communicably coupled to one or more remote sensors 301, (e.g., sensors located outside the sleep environment or outdoors) via a fourth communication link 294. In addition, the controller 500 may also be communicably coupled to one or more remote ISPs, computers, servers, databases, users, or the like via an external communication link 299, such as an Internet connection. One skilled in the art will recognize that the communications with the controller 500 and other components of the thermal system 100 may carried out according to any convenient communication technique, including standardized communication protocols, proprietary protocols, simple command/feedback signaling, etc. In addition, communications between one or more components of thermal system 100 may be based, at least in part, on processor architecture or requirements of other components used within the thermal system 100. For example, one or more communication links 291, 292, 293, 294, 299 may be configured to communicate on a network or using protocols associated with Android devices, iOS, CAN, LAN, IEEE 802.11, Internet communications, and other protocols like I2C, OneWire, SPI, etc.

One or more of these elements may be embodied as a plurality of components. For example, the processor 510 may include one or more processors, which may be dedicated to a particular control function of the thermal system. Examples of processors include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described throughout this disclosure. Also for example, the memory 520 may include a plurality of storage media, which may reside in different locations and may further include different memory technology from each other. Also for example, the communication port 530, may include may include a plurality of communication ports or channels, which may be configured to receive communications from several different sensors or sources, and according to a plurality of communication protocols. Also for example, the display 540, include may include a plurality of displays, which may each provide distinct information to a user or may include redundant information, and which may also be incorporated into a separate device (e.g., temporary use of another device's display via software application). Similarly, the user control 550, include a plurality of user interfaces, which may also be incorporated into a separate device and further combined with the display, such as a touch screen. Furthermore, each example may include a separate housing and/or power supply.

As illustrated, one or more components or elements of the controller 500 may be embodied as a single or integrate unit. The integrated unit may be housed in the housing 560, as shown. Alternately, an integrated unit may be on a circuit board or in an integrated circuit, for example. Here, the housing 560 contains the processor 510, the memory 520, the communication port 530 (further including an external communications port 531, and a removable unit including a combined display 540 and user control 550.

In other embodiments, the controller 500 may be embodied as a distributed control system. In particular, one or more elements of the controller 500 may be embodied as a discrete component outside the housing, combined with another element, distributed across or shared with other components of the thermal system 100, or any combination thereof. For example, all or part of the processor 510 and memory 520, respectively, may reside remotely from housing 560, such as on a mobile device, a remote server, a cloud network, etc. Also for example, the display 540 may be embodied as, or incorporated into an independent monitor such as a computer monitor, a display of a mobile device, or a TV/media screen. Also for example, the user control 550 may be embodied as, or incorporated into an independent device, such as a wearable device, a mobile device, or a remote control for another separate device, or embodied as an independent remote control.

As a processing system, controller 500 may execute software. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. Accordingly, in one or more exemplary embodiments, the functions or operation modes described may be implemented in hardware, software, firmware, or any combination thereof represented by memory 520, and If implemented in software, the functions may be stored on or encoded as one or more instructions or code memory on a computer-readable medium.

Computer-readable media includes computer storage media. Storage media may be any available media that can be accessed by a computer. Moreover, the computer-readable media may be physically located proximate, remotely, or mobile, relative to the sleep environment. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes compact disc (CD), laser disc, optical disc, digital versatile disc (DVD), and floppy disk where disks usually reproduce data magnetically, while discs reproduce data optically with lasers. Combinations of the above should also be included within the scope of computer-readable media.

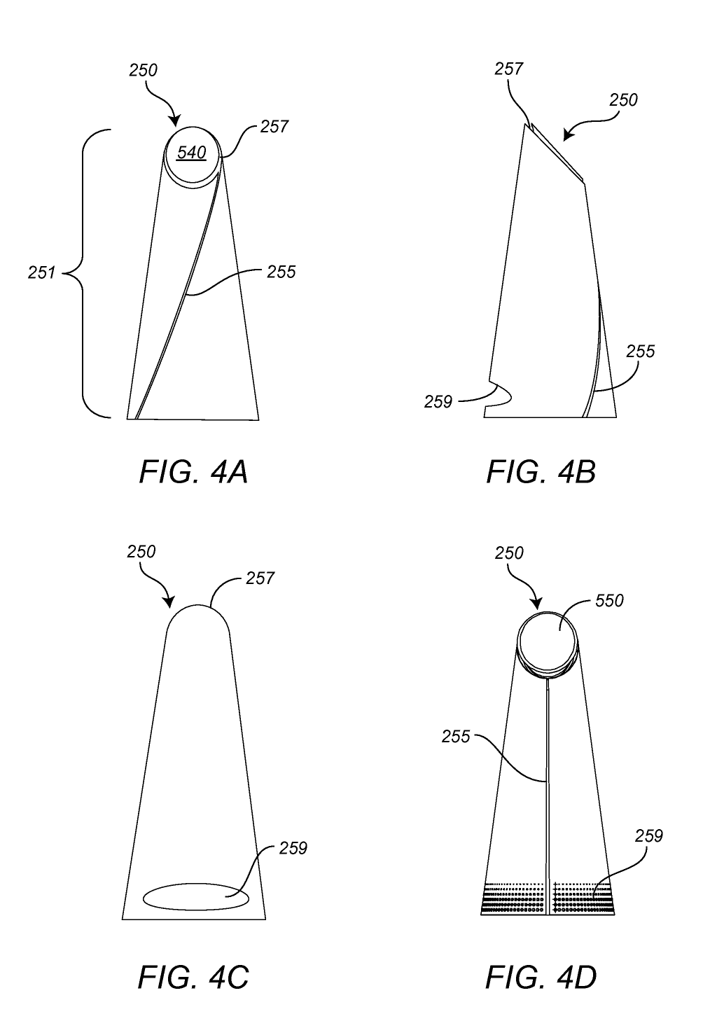

FIG. 3 schematically illustrates an exemplary heat exchanger of the thermal system of FIG. 1. In particular, the heat exchanger 200 is shown as a distributed system providing for a first heat exchange between a user and a coolant, and a second heat exchange between the coolant and the environment. Moreover, in this illustration, the first exchange is remote from the second exchange (e.g., performed in a separate module), which is conveniently represented by the break in coolant conduit 240. In addition, the heat exchanger 200 is shown at least partially integrated with an environment sensor 300, a user sensor 400, and the controller 500. Although the heat exchanger 200 is distributed into two units, it is understood that the heat exchanger 200 may be embodied as a single unit or as multiple units. Here, user sensor 400 is represented as including two sensors.

For reference, here and in other figures, the user interface 210 may be defined as having a head end 95 (corresponding to a head of a bed or user) and a foot end 96 (corresponding to a foot of a bed or user) opposite the head end 95. Also for reference, the user interface 210 may be further defined as having a vertical axis 97 (extending from the middle of the foot end 96 to the middle of the head end 95) and a horizontal axis 98, which perpendicularly bisects the vertical axis 97. Other orientations are contemplated, however.

According to one embodiment, the heat exchanger 200 may include the user interface 210, a cooler 220, a heater 230, and the coolant conduit 240. The coolant conduit 240 is configured to thermally couple the cooler 220 and the heater 230 to the user interface 210, routing a coolant path therebetween. Note, here, the cooler 220 and the heater 230 are combined as a pair of reversible Peltier cells, however, in other embodiments the cooler 220 and the heater 230 may be distinct units.

The user interface 210 is configured to conductively exchange heat between a user (e.g., while laying on top of the user interface 210) and a coolant (e.g., flowing through a coolant path). Likewise, the cooler 220 and the heater 230 are configured to exchange heat with the user interface 210 via a fluid coolant traveling therebetween through the coolant conduit 240. According to some embodiments, a fluid coolant may be a gas (e.g., air), a liquid coolant (e.g., water based, ethylene glycol based, silicone based, oil based). In other embodiments, at least one of the cooler 220 and the heater 230, may include operate with a gel coolant, a phase transition material, or a solid state device (e.g., Peltier device, thermal battery, heat sink/source).

As illustrated, the user interface 210 may include a thermal circuit 212 and a coolant conduit interface 214. The thermal circuit 212 is configured to conductively exchange heat between the user and a medium, such as a liquid coolant. In particular, the thermal circuit 212 may include a bound coolant passageway and have sufficient thermal conductivity to provide for both the conductive cooling and conductive heating at a physical interface with the user. In one embodiment, the coolant passageway of the thermal circuit 212 may be formed into another structure (e.g., channel formed into a sleeping pad). Alternately, the coolant passageway of the thermal circuit 212 may include a passageway in a dedicated flow structure (e.g., in tubing, piping, or some other fluid conduit). Advantageously, the thermal circuit 212 may be made from a material that flexes under a user weight distribution while maintaining an open flowpath. For example, the thermal circuit 212 may made from thermally conductive plastic tubing, flexible PVC tubing, and the like. According to one embodiment, the thermal circuit 212 may be made from a section of flexible PVC tubing having an outer diameter of 0.25″ (6.4 mm) and in inner diameter of 0.17″ (4.3 mm).

The coolant conduit interface 214 may generally include an inlet and an outlet, configured such that the thermal circuit 212 may begin and end at the coolant conduit interface 214. The inlet and an outlet may be proximate each other (e.g., panel-mounted), separated along one side of the user interface 210 (e.g., head end 95, foot end 96, side), or separated on different ends or sides of the user interface 210. The coolant conduit interface 214 may be configured to couple and form a flow path with the coolant conduit 240 via any conventional fluid couple (e.g., threaded, quick-release, etc.). In alternate embodiments, the thermal circuit 212 and the coolant conduit 240, may be combined or form a single unit, such that the coolant conduit interface 214 is merely a location where one transitions into the other. Where the coolant conduit 240 is detachable from the coolant conduit interface 214, the coolant conduit interface 214 may also include one or more self-sealing valves that seal or engage upon disconnection of the coolant conduit 240.

The thermal circuit 212 is configured to route the cooling media (e.g., flowing coolant) about the user interface 210 such that a user may be heated and cooled independent of sleeping position. In particular, the thermal circuit 212 may include a flow path that substantially traverses the user interface 210. The flow path begins and ends at coolant conduit interface 214, and may include serial portions, parallel portions, or any combination thereof. The thermal circuit may be further configured to exchange heat at a rate of at least 75 W with the user 10 or at least 1 W per kilogram of user 10, or at least 3 times the BMI of user 10, as discussed above.

According to one embodiment, the thermal circuit 212 may be composed of two or more independent and/or spatially separated units or circuits. This may helpful for cooling/heating different parts of the body differently. Moreover, this can also provide some thermal relief to the user's 10 skin due to the application of cooling and heating. For example, a specific area of user 10 skin can be aggressively cooled for a given amount of time, and then subjected to a more gradual cooling (or a brief warming) to reduce the thermal stress on the user 10. Also for example, some parts of user's 10 skin may be cooled aggressively, while others may have a more gradual cooling (or brief heating). These may provide for a faster cooling with improve thermal comfort, since stronger cooling can be applied. The same technique can be used during heating part of a thermal profile. Furthermore, in conjunction with feedback from user sensors configured to determine body position, separating the thermal circuit 212 may provide the additional benefit of efficiently heating/cooling only a part of skin that touches an independent unit or circuit of the thermal circuit 212, while leaving untouched (i.e., unused) portions off. Advantageously, this may minimize energy loss.

In addition, separating the thermal circuit 212 may provide for a thermal system where multiple users sleep in the same bed, and wherein the thermal system is able to provide different thermal profiles to each user as they move across the user interface 210 while sleeping. According to one embodiment, feedback from one or more user sensors may be indexed or otherwise associated with a particular user, so as to provide the different thermal profiles for each user.

According to the illustrated embodiment, the thermal circuit 212 may be laid out as a serial flow path traversing the user interface 210. In particular, the thermal circuit 212 may be laid out in a generally undulating pattern, biased in a vertical orientation, in a horizontal direction, or any combination thereof (e.g., diagonal, stepped, etc.). For example, the thermal circuit 212 may be biased in a vertical orientation (i.e., including a “vertical leg” that traverses the user interface 210 in a first direction, generally parallel to the vertical axis 97, turns around proximate one of the head end 95 or the foot end 96, traverses the user interface 210 in a second direction, again generally parallel to the vertical axis 97 and opposite the first direction, turns around proximate the other of the head end 95 or the foot end 96, and repeats so as to extend horizontally across the user interface 210). Advantageously, by configuring the thermal circuit 212 as a serial flow path or single path, as shown, a more flexible and/or thermally conductive conduit material may be used than in a parallel configuration, and/or a lower fluid pressure may be required, and the providing for greater comfort and/or more efficient operation. Also, by configuring the thermal circuit 212 in a vertical bias, the vertical legs my better correspond to the user's average sleep position, and the thermal conductivity to the user may be improved. In alternate embodiments, the thermal circuit 212 may be configured entirely or in part as a parallel system.

In addition, adjacent legs of the flow path may be positioned such that any pitch or spacing between is limited. In particular, each leg substantially traversing the user interface 210 may be sufficiently close to an adjacent leg to preclude or mitigate instances of a user fitting in the interstices (i.e., gaps between adjacent portions of the flow path flow path). For example, where the thermal circuit 212 is biased in a vertical orientation (as shown), the thermal circuit 212 may be further configured such that interstices in the horizontal direction (i.e., space between vertical channels) are approximately 2-3 inches (5-8 cm). Also for example, the interstices in the horizontal direction may be less than 6 inches (15 cm).). Also for example, the interstices in the horizontal direction may be between 18 inches (45 cm) and 6 inches (15 cm). Similarly, according to one embodiment where the thermal circuit 212 is biased in a vertical orientation, the thermal circuit 212 may be further configured such that “vertical legs” of the thermal circuit 212 are horizontally distributed at a density of 5 vertical legs per horizontal foot (30 cm). This may provide the benefit of a more continuous thermal coverage of the user interface 210, as the user will remain in direct contact with at least a portion of the user interface 210.

According to one embodiment, the user interface 210 may be configured to conductively warm and/or cool the user on a plurality of sides. In particular, the user interface 210 may include a base on which the user rests and a heat exchange cover or actively powered blanket that rests on the user. For example, the heat exchange cover may use a thermal circuit or a solid state heat pump, and be configured to conductively warm and/or cool the user in conjunction with the base underneath. Also for example, one or both of the base and cover may be limited to a single function, such as just cooling or just heating (e.g., controlled electric blanket). According to another embodiment the user interface 210 can include one or more thermal circuits in a pillow, a facial mask, a head cap, or any combination thereof, which too may be controlled by controller 500 and follow common or independent thermal profiles.

According to one embodiment, the user interface 210 may further include plurality of inflatable bladders or chambers configured to promote skin integrity and prevent skin breakdown. In particular, the user interface 210 may include air-filled channels that alternately fill and empty to keep bearing weight off bony prominences of the user. Moreover, the thermal system 100 may include an air system configured inflate and deflate the inflatable bladders via the controller 500. The may be beneficial for immobilized or weak patients who are unable to shift their weight frequently and provide for a more comfortable sleep.

According to one embodiment, the cooler 220 and the heater 230 may be combined. In particular, a heat pump module 250 may be configured to switchably operate as either the cooler 220 and the heater 230, or otherwise combining the two as a single unit. For example, the heat pump module 250 may include any combination of one or more reversible heat exchangers, heat pumps, etc. Also for example, the heat pump module 250 may include one or more reversible heat pumps, such as solid state heat pumps, compression heat pumps, absorption heat pumps, or any combination thereof.

As discussed above, the heat exchanger 200 may be a distributed system. In particular, the heat exchanger 200 may be configured for a first heat exchange between a user and a coolant (e.g., in a bed), and a second, remote heat exchange between the coolant and the environment (e.g., on the floor). For example, here, the heat exchanger 200 is configured as having the user interface 210 coupled to a separate heat pump module 250 via the coolant conduit 240, wherein a coolant may be pumped or otherwise communicated therebetween. In other embodiments, the heat exchanger 200 may be configured as the user interface 210 switchably coupled to the cooler 220 via a first coolant conduit and to the heater 230 via a second coolant conduit. This may be beneficial where the functionality of at least one of the cooler 220 and the heater 230 is provided by an independent device, such as a component of a home HVAC system.

As in the illustrated embodiment, the heat pump module 250 may include both the cooler 220 and the heater 230, as well as a coolant reservoir 252, a coolant pump 254, and a coolant conduit interface 256. As above, each feature may be a single unit or a plurality of units. The coolant reservoir 252, coolant conduit interface 256, the cooler 220, and the heater 230 may be plumbed together, and the coolant pump 254 may be configured in any convenient or efficient manner to energize a coolant to propel it through the heat exchanger 200. For example, here, the heat pump module 250 includes a coolant pump 254 upstream of the cooler 220 and heater 230, and another coolant pump 254 downstream of the cooler 220 and heater 230. It should be understood, however, that the pumping or transportation of the coolant may be energized at one or more locations throughout the heat exchanger 200 (e.g., in the heat pump module 250, the user interface 210, the coolant conduit 240, or any combination thereof). Moreover, the various components or features of the heat pump module 250 may be concealed or housed in an esthetically pleasing outer shell.

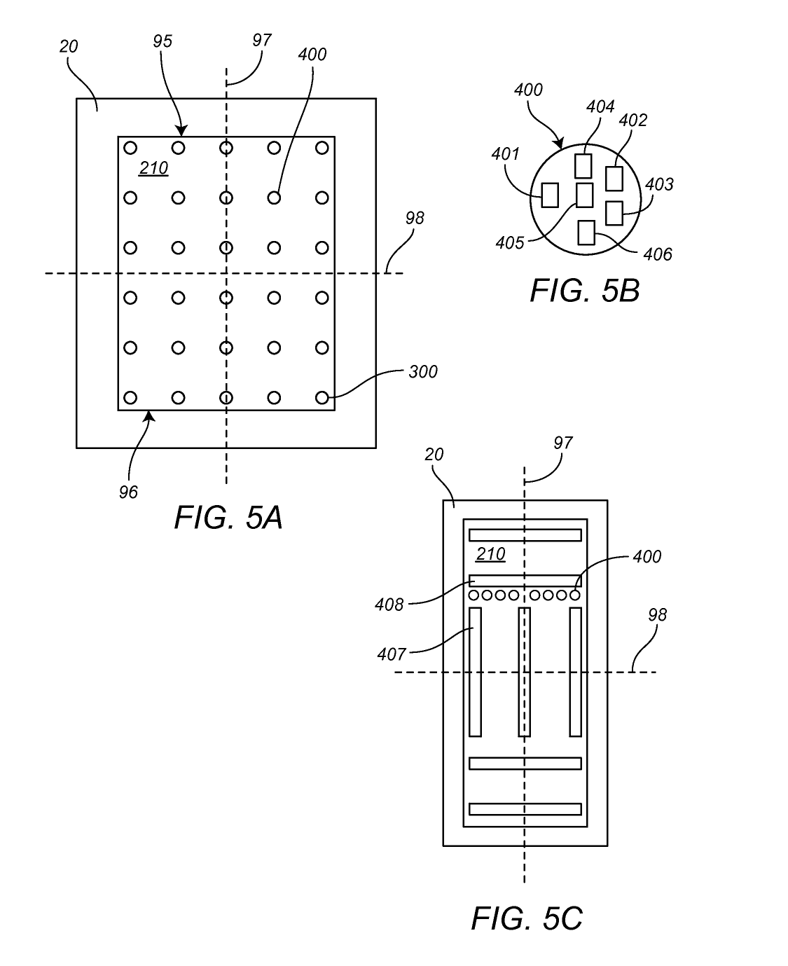

FIGS. 4A-4C illustrate various views (front, side, and back, respectively) of one exemplary embodiment of the heat pump module of FIG. 3. In particular, the heat pump module 250 is illustrated according to one industrial design, which sits flush on a base or floor. As above, the heat pump module 250 may include an outer shell or housing 251 configured to house and/or conceal one or more components of the heat pump module 250. The housing 251 may include an air inlet 257 and an air exhaust 259 configured to provide ambient air to and from a flowpath within the heat pump module 250. Here, the ambient air may flow into a flared or louvered air inlet 257 at the top of the heat pump module 250 and exit the heat pump module 250 from the air exhaust 259 at the back of the heat pump module 250, and generally away from the user. According to one embodiment, the airflow may be in the opposite direction, such that the air inlet 257 and the air exhaust 259 are functionally reversed.

FIG. 4D, illustrates a front view of another exemplary embodiment of the heat pump module of FIG. 3. Here, the air exhaust 259 is configured as an array of exhaust holes circumscribing a lower portion of the housing 251. The array of exhaust holes of the air exhaust 259 progressively increase in diameter with each ring, and each ring may substantially circumscribe the heat pump module 250 in a substantially uniform manner. According to one embodiment, the airflow may be in the opposite direction, such that the air inlet 257 and the air exhaust 259 are functionally reversed.

According to one embodiment, the air inlet 257 may be angled so as to provide a display surface toward the user when the air exhaust 259 is pointed away. This surface of the air inlet 257 may be configured as a user control 550 (see FIG. 4D) and/or a display 540 as discussed above, or as a user control interface 253, as discussed below. Moreover, the entire housing 251 may be configured as a user control, such as a touch control.

As illustrated, the heat pump module 250 may also include a light source 255. The light source may include one or more light elements, such as LEDs, incandescent bulbs, light pipes, fluorescent lights, photoluminescent materials, etc. Here, the light source 255 is shown curving down the height of the housing 251. In FIG. 4D, however, the light source 255 is shown as a variation, running vertically down the height of the housing 251. According to one embodiment, the light source 255 may be configured as a user control.

Moreover, the light source 255 may vary in color. In particular, the color of the entire light source 255 may be variable, or portions of the light source 255 may just vary from one another. For example, the light source 255 may vary in color based on a user selection. Also for example, the light source 255 may vary in color based on a time, such as amber light early in the sleep cycle and deep red light later in the sleep cycle. As such, the light source 255 be customizable to a particular user for both esthetics and melatonin production.

In addition, the light source 255 may have varying intensity. The varying intensity of the light source 255 may be provided by separate light elements, by variable intensity light elements, or any combination thereof. For example, the light source 255 may include a low-output light source, such as a night light or dim light that is not disruptive to sleep but has sufficient luminosity to identify the location of the heat pump module 250 in the dark. Also for example, the light source 255 may include a light source having sufficient luminosity to provide for user visibility, such as a room illumination or spot illumination.

In addition, the light source 255 may be configured to be visible from multiple directions. As illustrated, the light source 255 may be configured such that it spans at least 90 degrees about the heat pump module 250. In addition, the light source 255 may extend substantially the height of the heat pump module 250, or otherwise indicate an elevation profile of the heat pump module 250 visible in the darkness. For example, the light source 255 may be distributed or otherwise extend from a first elevation at a first radial, proximate the top of the heat pump module 250, down to a second elevation at a second radial, proximate the bottom of the heat pump module 250, wherein the second radial is swept at least 90 degrees from the first radial.

According to one embodiment, the light source 255 may be configured such that it is visible from 360 degrees about the heat pump module 250. For example, the light source 255 may be positioned atop the heat pump module 250, such as at an apex. Also for example, the light source 255 may circumscribe the heat pump module 250 at any elevation. Also for example, the light source 255 may begin to circumscribe the heat pump module 250 at a first elevation, and terminate at a second elevation, such as a spiral shape. It is understood that the heat pump module 250 may have a curved footprint (as illustrated), a rectilinear footprint, an irregular foot print, or any combination thereof.

According to one embodiment, the light source may be controlled automatically, manually, or a combination thereof. In particular, the light source 255 may be configured to emit light in response to ambient lighting conditions, time of day, user presence (e.g., proximate motion detection, user activation, thermal system activation, smart home triggering, etc.), sound, sleep state determination, or any combination thereof. For example, the controller 500 may automatically control the light source 255 as preprogrammed or programmed by a user. Also for example, a user may activate or program the light source 255 via the user control 550 of the controller 500. According to one embodiment, the display 540 of the controller 500 may function as the light source 255

Returning to FIG. 3, according to one embodiment, the cooler 220 and the heater 230 may be combined as a Peltier device 270. In particular, when controlled by the controller 500 to receive power in a first polarity, the Peltier device 270 may conduct heat into the coolant, and when powered in the opposite polarity, the Peltier device may conduct heat away from the coolant. In each case, a “heated” or “cooled” coolant can be pumped, sucked, or otherwise delivered to the user interface 210 for further heat exchange with the user. In this way, the heat exchanger 200 may be greatly simplified both in number of components and in operation. According to one embodiment, the Peltier device may be rated at 10 A at 12 VDC, or equivalent in cooling/heating power. According to another embodiment, the Peltier device output may be rated at approximately 240 W, at least 200 W output, or between 200 W and 300 W. According to another embodiment, the Peltier device may be rated at 250 W-500 W input power.

As illustrated, the Peltier device 270 may include a liquid radiator 272 thermally coupled to one side of a Peltier cell 274 and an air radiator 276 (e.g., fin radiator) thermally coupled to the other side of the Peltier cell 274. The Peltier device 270 may further include a fan or blower 278 configured to pass air over or through the air radiator 276, convecting heat away. Moreover, the heat pump module 250 may include a flowpath 258 configured duct air to and from the blower 278. As above, each feature may be a single unit or a plurality of units.

According to one embodiment, the liquid radiator 272 provides direct contact between the coolant liquid and the Peltier cell 274. In particular, a flow path of the liquid radiator 272 may be at least partially bounded by a surface of the Peltier cell 274. For example, the liquid radiator 272 may include an coolant inlet, a coolant outlet, and a sealing perimeter, and may be bound on opposing sides by Peltier cells 274 (e.g., ceramic plates of the Peltier element or cell) wherein the sealing perimeter prevents coolant from escaping the bounded area, other than through the coolant inlet and coolant outlet.

According to another embodiment, the liquid radiator 272 may direct the coolant along a path for optimized thermal exchange between the Peltier cell 274 and the coolant. For example, the liquid radiator 272 may include one or more vanes, guides, baffles, and the like, which are configured to direct the liquid coolant about an optimized thermal exchange path within the liquid radiator 272. Alternately, the liquid radiator 272 may not direct the liquid to follow any specific path between inflow and outflow directions.

According to one embodiment, the Peltier device 270 may be configured such that the liquid radiator 272 has Peltier cells 274 on opposing sides, and each Peltier cell 274 has an air radiator 276 opposite the liquid radiator 272. For example, were the liquid radiator 272 has two opposing flat heat exchange surfaces, the Peltier device 270 may include two Peltier cells 274, with one on each surface. Also for example, the Peltier device 270 may include four Peltier cells 274, with two on each opposing side. According to one embodiment, each of the two or four Peltier cells 274 may be rated at 10 A at 12 VDC. Also for example, each of the two or four Peltier cells 274 may be rated at 5 A at 24 VDC. According to another embodiment the Peltier device 270 may be configured to have one or more Peltier cells 274 on only one side of the liquid radiator 272, and wherein the one or more Peltier cells 274 of rated at a similar or higher power as the examples above.

Also for example, were the liquid radiator 272 has a cylindrical heat exchange surface, the Peltier device 270 may include an annular Peltier cell 274 circumscribing the liquid radiator 272. Other geometries are contemplated. Similarly, the air flow system may take many geometries. In particular, the air flow system may be adapted for both esthetics and performance of the heat pump module 250. For example, here the housing 251 of the heat pump module 250 includes a plurality of air inlets 257 and a single downward-facing air exhaust 259, and the blower 278 includes fans upstream and downstream of the air radiator 276. In another embodiment, the air flow may circulate in the opposite direction. Moreover, in yet another embodiment, the blower 278 may be reversible such that the air flow may circulate in both directions.

The heat pump module 250 may also include or otherwise incorporate one or more of a user control interface 253, a user control 550 and a display 540. As illustrated, a combined user control 550 and display 540 may be removably attached to the user control interface 253. In this configuration, the combined user control 550 and display 540 may have the benefit of serving as a removable remote control for the thermal system.

According to one embodiment, the user control interface 253 may be configured to charge an energy storage of the user control 550 and/or display 540 when seated into or otherwise engaged with the user control interface 253. Moreover, in embodiments where an independent device (e.g., smart phone, tablet, or other device loaded with interfacing software) operates as one or more of the controller 500, the user control 550, and the display 540, the user control interface 253 may be configured as a charger for the independent device. For example, the user control interface 253 may include a device adapter or universal-type interface (e.g., microUSB, induction charge interface, etc.), and may be further configured as a wired or wireless charger.

According to one embodiment, the heat exchanger 200 may be communicably coupled to an independent heat exchanger. In particular, the controller 500 may communicate via an external communication link 299 (see FIG. 2) with the independent heat exchanger and operate the independent heat exchanger in coordination with the heat exchanger 200. For example, the controller 500 may wirelessly communicate with a heater or air conditioner to enhance the heating or cooling of the user. Likewise, the controller 500 may communicate with other independent devices, such as humidifiers, room lighting, colored lighting (e.g., for melatonin production), multimedia devices, electronic blinds, smart home controller, alarm clock, and other devices that may be coordinated with the thermal system to, enhance the sleep environment, reduce sleep disruptions, and/or aid in scheduled waking of the user.

According to one embodiment, the controller 500 may operate a heater or air conditioner inversely from the user interface 210. In particular, the controller 500 may operate a room heater or an air conditioner to provide a warm air temperature while cooling the user or a cool air temperature while warming the user. The inventor has further discovered it may benefit the user to invert the ambient or room air temperature while conductively managing the user's skin temperature with the user interface 210. This may aid in maintaining a body temperature despite the thermal exchange with the heat exchanger, or may otherwise provide for a more comfortable sleep. In the case of multiple users sharing the same sleep environment, the controller 500 may operate the heater or air conditioner to provide the warmer air temperature, but which is common to all the users.

According to one embodiment the thermal system may be configured to direct its own thermal exhaust toward the user instead, or in combination, with the heater or air conditioner described above. For example, the heat pump module 250 may direct its exhaust upward and or toward the user, such as by reversing a blower fan direction. This may beneficially recycle the energy imparted into its exhaust, or otherwise provide for more efficient operation. Moreover, the thermal change of the ambient air may be slight and need not be on the order of the user interface 210.

Returning to FIG. 1, as discussed above, the illustrated environment sensor 300 may represent one or more environment sensor(s) 300 configured to sense and communicate environmental conditions associated with the sleep environment. The one or more environment sensor(s) 300 may sense directly or indirectly. The one or more environment sensor(s) 300 may be located proximate or remote from one another. Moreover, one or more environment sensor(s) 300 may be located proximate or within a component of the thermal system 100. For example, one or more environment sensor(s) 300 may be integrated with or otherwise fixed to the heat exchanger 200 or the controller 500. Furthermore, one or more environment sensor(s) 300 may be located remotely from a component of the thermal system 100. For example, one or more environment sensor(s) 300 may be deployed about the sleep environment, fixed to the user 10 (e.g., as a wearable device such as wearable user sensor 410), located outdoors, or at the termination of an independent communication link (e.g., Internet link, wireless link, telecommunications link). According to one embodiment, the controller 500 may operate the heat exchanger 210 to thermally condition the sleep environment based, at least in part, on current and forecast weather conditions, temperature, humidity, dew point, air pressure, pollen count, smog index or air quality, etc.

An environment sensor 300 may directly measure a metric or attribute associated with the sleep environment. For example, environment sensor 300 may include temperature sensors, humidity sensors, light sensors, sound sensors, etc. These sensors may directly measure their respective metrics and communicate the measured data or further process the data (e.g., sample, compare, digitize, combine, issue commands, alert, etc.).

In addition, an environment sensor 300 may indirectly measure or otherwise derive a metric or attribute associated with the sleep environment. In particular, metrics or attributes associated with the sleep environment may be determined based on the time of day, its location, the date/season/weather, or characteristics of the sleep. For example, environment sensor 300 may include a device or sensor configured to measure or determine time (e.g., clock or timer), location (e.g., via GPS, digital maps, IP address), date (e.g., calendar), external weather conditions (e.g., NOAA Internet feed, barometer), room profile, etc. As above, these sensors may determine their respective metrics and communicate or further process the data. In addition, these sensors or devices may be incorporated into a processor of the controller 500, particularly where information is derived from a remote source such as over the Internet.

Also as discussed above, the illustrated user sensor 400 may represent one or more user sensor(s) 400 configured to sense and communicate at least one metric of the user 10. The one or more user sensor(s) 400 may sense directly or indirectly. The one or more user sensor(s) 400 may be configured to sense and communicate environmental conditions proximate the user 10. In particular, the one or more user sensor(s) 400 may be located proximate or remote from one another. Moreover, one or more user sensor(s) 400 may be located proximate or within another component of the thermal system 100. For example, one or more user sensor(s) 400 may be integrated with or otherwise fixed to the user interface 210 or the heat pump module 250 (FIG. 4).

Furthermore, one or more user sensor(s) 400 may be located remotely or may be otherwise removable from a component of the thermal system 100 and/or the sleep environment. For example, one or more user sensor(s) 400 may be deployed about the sleep environment, fixed to the user 10 (e.g., such as wearable user sensor 410), located outdoors, or at the termination of an independent communication link (e.g., Internet link, wireless link, telecommunications link), such as a sensor of an independent system that is configured to communicate data regarding the user 10 to the thermal system 100 over the independent communication link.

The metric or attribute associated with the user 10 may be measured directly with nonintrusive or minimally intrusive techniques that are compatible with sleep. Moreover, the user sensor 400 may sense at least one metric used to make a user sleep determination. To illustrate, the one or more user sensors 400 may include accelerometers and temperature sensors, as well as sensors for user presence detection, body position/motion, pulse (heartbeat), breathing, muscle tone/signaling, blood oxygenation, brainwave activity (EEG Electroencephalograph), skin conductance/skin humidity, and other sensors commonly associated with measuring the sleep state. A temperature sensor may include, for example, an eletromechanical temperature sensor, an IR sensor, or heat camera. The user sensor 400 may also measure the differential temperature between body core and extremities, for example, by using a strap, grid, computation techniques.

According to one embodiment, the user sensor 400 may include a pulse oximeter or pulse-ox sensor, including at least two lights of different color (e.g., at least one red LED and one infrared (IR) LED) and a light sensor. A person skilled in the art will recognize how to construct a pulse-ox based on a light sensor and plural LEDs. In addition, each light of the pulse-ox sensor may individually controlled. In this way, the IR light may be used to detect user presence by detecting whether the IR light and light sensor detect a heartbeat, and once a user presence is detected, the red light may be turned on. Since IR light is invisible, the user detection may be may made without disrupting the user 10 while sleeping or attempting to sleep, then once the user is detected, the visible red light may be turned on to determine, inter alia, blood oxygenation. Advantageously, IR light can also pass through thin fabric such as bed sheets and pajamas, and moreover, IR light is not visible.

In alternate embodiments, the user sensor 400 may include one or more additional sensors capable to detect user presence, such as a temperature sensor and/or a capacitive sensor. The one or more additional sensors may be used to detect user presence instead of the pulse-ox sensor, providing for the use of a pulse-ox sensor that does not require the independent light control described above. Alternately, the one or more additional sensors may be used to detect user presence in addition to the pulse-ox sensor described above, providing for redundancy before turning on the visible light, which may potentially be disruptive to sleeping or falling to sleep.

According to one embodiment, the visible light of the pulse-ox sensor may be controlled based on the body position of the user. In particular, the visible light of the pulse-ox sensor may be limited to where the user is unlikely to see it. For example, the controller 500 may be configured to determine the position, particularly the center of mass of the user based on feedback from one or more user sensors 400. Then, the controller may limit operation of the visible (red) light of the pulse-ox sensor to that location, which indicates both the presence of the user's body and the absence of the user's head (eyes). In alternate embodiments, the pulse-ox sensor(s) may be limited to locations on the user interface 210 where the user's body is likely and the user's head is unlikely, such as the middle region of the user interface 210 or the bottom two-thirds of the user interface 210, for example.

According to another embodiment, once the user presence is detected, that specific user sensor 400 may activate the other sensors to collect every user measurement that is possible given the set of sensors implemented. Known computational techniques may be used to compute the most likely body configuration (e.g., extended, fetal, side, back, front, diagonal, etc.), given the position of a given user sensor 400 and the position of user sensor 400 that most strongly detects or otherwise locates a beating heart. Similarly, the most likely body configuration may be determined by detecting a change of temperature received, for example by motion of the body of user 10.

In another embodiment the user 10 may communicate with the controller 500 via hand tapping or hand swiping near any user sensor 400. This may minimize user sleep disruption (compared to communicating via a user control located, for example, on the heat pump module 250) and provide a simple and easy communication channel for user 10. For example, if the user 10 is too cold, the user 10 may tap on heat exchanger 210 or mattress as a user input, and the controller 500 may responsively adapt the thermal-comfort profile to provide a more comfortable (warmer) sleep environment.

According to one embodiment, the controller 500 may communicate back to the user 10 via auditory signal such as tone, music, natural sound, etc. Moreover, a different sequence of taps or swipe could indicate that the user 10 is too warm, and the controller 500 may adjust the thermal-comfort profile, and play a different auditory signal as an acknowledgement. Alternatively, the same single tap, or sequence of taps, or swipe could indicated the controller 500 to switch between cooling/warming/neutral and communicate back to the user via different auditory signal. In this way the user could select the current desired condition with minimal motions and distractions.

As above, metrics or attributes associated with the user 10 may be metrics or attributes directly. User presence, body position, body motion, pulse and breathing may be measured, for example, with a piezoelectric sensor (PVDF material), capacitive sensor, wearable near field communications device, an accelerometer, an IR sensor, force or pressure sensor, or heat camera. Muscle tone/signaling may be measured, for example, with a conductive pad, bracelet, or direct electrode. Blood oxygenation may be measured, for example, with a transmissive pulse oximeter, a reflectance pulse oximeter, or an IR light, a LED, and photodetector. Other metrics or attributes may be measured, for example, with user sensors 400 including humidity sensors, light sensors, sound sensors, etc. These sensors may directly measure their respective metrics and communicate the measured data or further process the data (e.g., sample, compare, digitize, combine, issue commands, alert, etc.). Advantageously, these sensors may provide feedback to improve thermal comfort detection algorithm, provide the ability to detect which body part needs localized warming/cooling, or to track user sleeping position, to name a few.

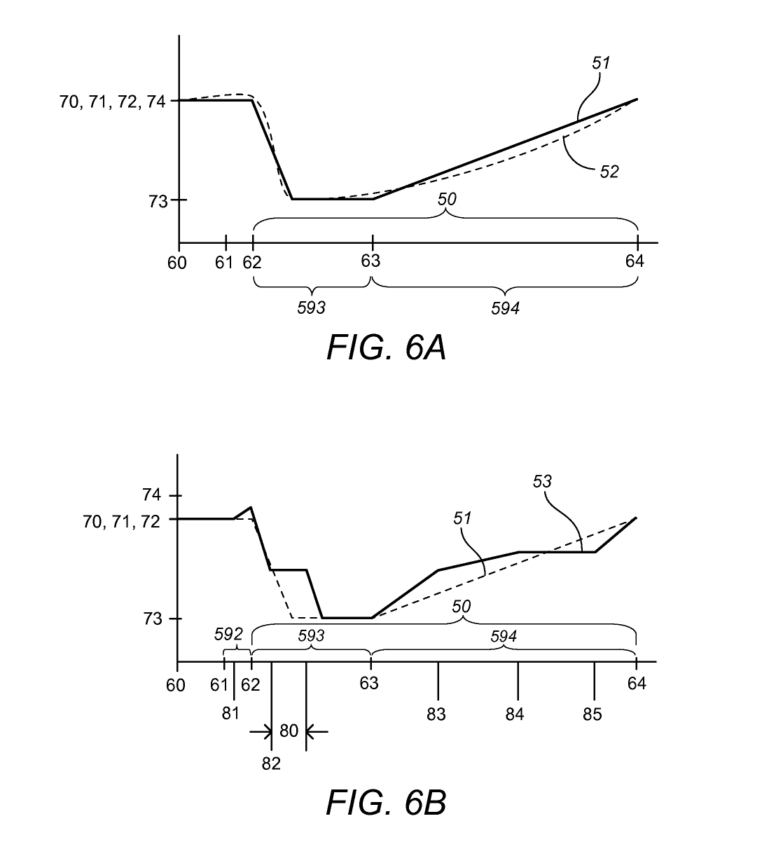

According to one embodiment, a plurality of sensors may be combined. In particular, a single sensor may perform multiple functions. For example, user sensor 400 may include sensitive motion sensors like a piezoelectric sensor or an ultrasensitive accelerometer configured to measure coarse metrics such as presence, body position, body motion, as well as fine metrics such as pulse, breathing, distinguishing variations between users, and identifying users from these metrics or other variations of sensed metrics. These sensors may also be used to directly detect sleep phases with delay differential equations. Likewise, a single sensor unit may contain multiple sensor types. In particular, FIG. 5B schematically illustrates an exemplary sensor unit of the thermal system of FIG. 1. For example, here, the user sensor 400 may combine an accelerometer 401, a temperature sensor 402, a capacitive sensor 403, an IR light 404, one or more LEDs 405 of different color, and a photodetector 406. By combining sensors, benefit may include reduced complexity, reduced costs, reconfigurability by the controller 500, and bus or daisy chain communications.

In addition, metrics or attributes associated with the user 10 may be measured indirectly. In particular, metrics or attributes associated with the user 10 may be determined based on the time of day, his location, the date, her schedule, the season/weather, or characteristics of the user 10. For example, user sensor 400 may include device or sensor configured to measure or determine time (e.g., to determine length of sleep, thermal-comfort profile, etc.), the date or schedule (e.g., to determine likely body state, sleep cycle requirements, etc.), location (e.g., to determine thermal system activation, likely body state, etc.), date/external weather conditions (e.g., to determine likely body state, sleep cycle requirements, etc.), user profile (e.g., historical data, user preferences, or physical characteristics such as weight, BMI, sex, and the like). As above, these sensors may determine their respective metrics and communicate or further process the data. In addition, these sensors or devices may be incorporated into a processor of the controller 500, particularly where information is derived from a remote source such as over the Internet.

FIG. 5A schematically illustrates an exemplary sensor array configuration of the thermal system of FIG. 1. Here, the user interface 210 is shown resting on top of a bed 20. A plurality of sensors are shown distributed across the user interface 210 between the head end 95 and the foot end 96 as an evenly distributed array. While the sensors are configured as a regular array, symmetric with the vertical axis 97 and the horizontal axis 98, in other embodiments, the sensors may be irregularly and asymmetrically distributed. In one embodiment, the sensor array may include a single row of user sensors 400, each separated by the 3-4 inches (7-10 cm).

The sensors may include environment sensors 300, user sensors 400, or a combination of both. The user sensors 400 may be configured to cover a sleepable area of the bed, while the environment sensors 300 may be positioned at extremities of the user interface 210. Moreover, the user sensors 400 may be concentrated or more densely populate portions of the user interface 210 where a majority of user's body is likely to be or “high traffic areas” of the sleeping area. For example, the density of user sensors 400 may decrease with their distance from one or both of the vertical axis 97 and the horizontal axis 98.

According to one embodiment, the array of the user sensors 400 may be arranged to detect the user regardless of position or orientation on the user interface 210. In particular, the user sensors 400 may substantially traverse the user interface 210 with sufficient density to preclude or mitigate instances of a user fitting in the interstices or otherwise go undetected. For example, the user sensors 400 may be positioned about the user interface 210 so as to extend to at least 12 inches (30 cm) from an edge of the sleeping area or bed 20. Also for example, the user sensors 400 may be positioned about the user interface 210 so as to have a density of at least 1 sensor per square foot (0.09 sensor per square meter) or a spacing that is no more than 12 inches (30 cm) from an adjacent user sensor 400. Moreover, the user sensors 400 may be positioned about the user interface 210 with a greater density above the horizontal axis 98 than below the horizontal axis 98. For example, the user sensors 400 may be positioned about the user interface 210 so as to have a density of at least 1.25 sensors per square foot (0.12 sensor per square meter) or a spacing that is no more than 10 inches (25 cm) from an adjacent user sensor 400 above the horizontal axis 98 and a density of at least 1 sensor per square foot (0.09 sensor per square meter) or a spacing that is no more than 12 inches (30 cm) from an adjacent user sensor 400 below the horizontal axis 98 or a spacing that is no more than 12 inches (30 cm) from an adjacent user sensor 400 in any direction.

According to another embodiment, the user sensor 400 may include one or more sensor strips. FIG. 5C schematically illustrates an exemplary sensor configuration including sensor strips of the thermal system of FIG. 1. The sensor strips may include sensitive motion sensors like a piezoelectric sensor or an ultrasensitive accelerometer, or capacitive sensors or touch sensors. In addition to simultaneously detecting different metrics such as breathing rate and heart rate, this configuration of user sensor 400 may both detect the user regardless of position or orientation on the user interface 210, and greatly simplify the thermal system by reducing the number of communication links to the user sensors 400.

As illustrated the user interface 210 may include a vertical sensor strip 407 and a horizontal sensor strip 408. In particular, a vertical sensor strip 407 may be more adapted to detect metrics associated with a body core (e.g., temperature, breathing, heart rate, sleep phase, etc.) and the horizontal sensor strip 408 may be more adapted to detect metrics associated with the body's extremities (e.g., presence, position, orientation, extension, motion, etc.). For example, as illustrated, the user interface 210 may include a series of vertical sensor strips 407 distributed horizontally across the user interface 210, and which may intersect the horizontal axis 98. Also for example, the user interface 210 may include one or more horizontal sensor strips 408 distributed horizontally across the user interface 210, and which may intersect the vertical axis 97. Moreover, the one or more horizontal sensor strips 408 may be located below the horizontal axis 98, as well as below the vertical sensor strips 407 (corresponding to a leg position). This may be beneficial in detecting whether the user 10 has assumed a fetal position (indicative of being cool) or extended orientation (indicative of being warm). Furthermore, the one or more horizontal sensor strips 408 may be located above the horizontal axis 98, as well as above the vertical sensor strips 407 (corresponding to a head position). Head position may be used to determine user position, as well as to interpret other sensor feedback.

As illustrated, the user interface 210 may be smaller than the sleeping area or bed 20. Here, the user interface 210 is centered on the bed 20 and is configured to substantially cover its sleeping area. As such, user sensors 400 (and an underlying thermal circuit) may extend up to the edges of the user interface 210.

According to one embodiment, the controller 500 may track user sleeping position. In particular, position feedback of the user sensors 400, such as thermal or presence data, may be recorded in addition to being used for heat exchanger operation. The data may be processed in the controller 500 or communicated to a remote processor so as to determine sleep metrics such as sleeping position, movement, and correlation with other sleep data. In this way, the user's sleep data may be quantified over time and may be used in learning algorithms to improve the operation of the thermal system or other sleep-related reporting.

According to one embodiment, the user interface 210 may be configured to conform to a standard bed size. For example, the user interface 210 may include a pad sized with substantially the same plan dimensions as a twin, full, queen, king, or California king-sized mattress. Alternately, the user interface 210 may include a pad sized to an anticipated sleeping area, rather than a bed top surface. In particular, the user interface 210 may be sized slightly less than a standard bed size, so as to include inward offset of 6-12 inches (15-30 cm). This may be beneficial in reducing conflicts with fitted sheets and lowering material costs, while optimizing thermal coverage to the anticipated actual sleep environment. Similarly, the user interface 210 may include a pad sized in non-standard dimension altogether, such as for placement on chairs/floor, benches, etc.

In other embodiments, the user interface 210 may be configured to cover only a portion of sleeping area, such as one side of the vertical axis 97. In particular, the user interface 210 may be configured such that one size may be used on a plurality of standard sized beds, or so that a second user interface 210 may be used for a second user. In this embodiment, the user sensors 400 of each user interface 210 may increase density proximate the vertical axis 97. In this way, the user sensors 400 may provide more refined feedback indicative of one user crossing the vertical axis 97. In this way, each controller or a single common controller may distinguish the first user from the second user.

According to one embodiment, the user sensors 400 of a first user interface 210 may communicate with the controller of a second user interface 210. In particular, feedback regarding a first or second user on the first user interface 210 may be provided to a first controller 500, and vis versa. For example, the first user interface 210 and second user interface 210 may be configured to operate as a single unit. Also for example, the first user interface 210 and second user interface 210 may be configured to share user feedback. Moreover, the controller or controllers may collect sufficient feedback data to distinguish a first user from a second user. For example, the controller 500 may refine data collection parameters, increase sample frequency, or collect feedback from additional types of user sensors present, or other techniques to identify each user. This may be particularly beneficial, for example, to make a sleep determination of a second user who is at least partially on the second user interface 210.

In other embodiments, multiple users may share the same user interface 210 at different times, for example, by trading places on a bed with a plurality of user interfaces 210, or sleeping at different times in a bed with one or more user interfaces 210. In this case, the controller 500 may be configured to identify each user from feedback from one or more user sensors 400, historical data, or direct identifying input from the particular user (e.g., via a user control 540, an independent device such as a smartphone, a communicably coupled server, other means). This may be particularly beneficial in applications like hotels or cruises, where different users can sleep in the same bed at different time.

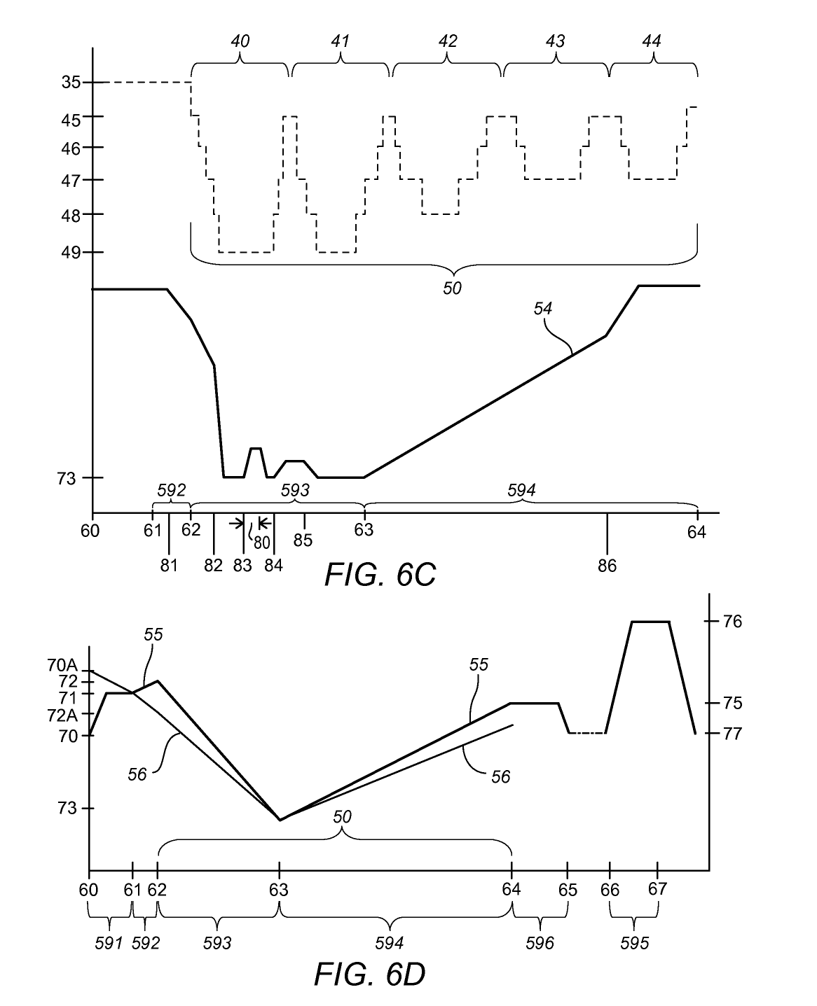

FIGS. 6A-6D illustrate various exemplary thermal-comfort profiles and exemplary operation modes of the controller of the thermal system of FIG. 1. Each operation mode may include a characteristic performance shape or perform a role within a thermal-comfort profile. In general, FIGS. 6A-6D each show a measured temperature over time. In particular, each figure represents one or more exemplary thermal-comfort profiles 51-56, which are associated with the operation of the heat exchanger 200. Also, each figure includes a representative sleep cycle 50 in its time scale, which broadly refers to the period between falling asleep to waking up. The sleep cycle 50 may be a duration of time that is independently predefined (e.g., estimated or preprogrammed), or empirically determined, for example, based on sensor feedback.

While the thermal-comfort profiles 51-56 are conveniently shown as continuous curves occurring over the sleep cycle 50, one or more operation modes illustrated may be discontinuous, or operated independently from its position within illustrated thermal-comfort profile or sleep cycle 50. Moreover, one or more illustrated operation modes may occur before or after the sleep cycle 50 without encompassing any sleep cycle at all.

For clarity, the illustrated sleep cycle 50 is common to each figure. However, the sleep cycle 50 may vary, which may be based on the configuration of the controller 500 and/or user requirements. In particular, sleep cycle 50 may be predetermined based on a presumed or standard sleep cycle (e.g., 8 hours), a desired or specified sleep cycle (e.g., number of hours/minutes inputted by the user), an actual user sleep determination (e.g., determined via direct measurement or historical user data) followed by an actual wake determination, or any combination thereof.

According to one embodiment, a user sleep determination may be made by designation or constructively determined. For example, a constructive user sleep determination may be conveniently “designated” as 1, 10, or 20 minutes from a system activation or a user entry into the bed, regardless of whether or not the user is actually asleep. Advantageously, the inventor has discovered that an “early” initiation of the sleeping mode may assist the user in falling asleep. According to another embodiment, the sleep determination may be empirically derived or approximated for a user, for example, as being an average time it takes for the user to fall asleep. According to another embodiment, the sleep determination may be determined substantially in real time. For example, the sleep determination may be computed based on heart rate, heart rate variability, breathing rate, breathing rate variability, blood oxygenation, temperature, differential temperature between body core and extremities, brainwave activity, body motion, muscle tone/signaling, skin conductance, and other nonintrusive or minimally intrusive techniques used to determine a sleep state.

In addition, sleep cycle 50 may be generalized for a nonspecific user or for a class of users (e.g. based on age, general health, acute health condition, etc.), or may be adapted to a particular user. Furthermore, sleep cycle 50 may be varied over time, for example, in response to one or more users' feedback (e.g., provided by a user, collected historically, etc.), in real time (e.g., empirical measurement of sleep phases), in response to learning algorithms, in response to transitory conditions (seasons, weather, health conditions), or in response to other modifying factors.

For clarity, each thermal-comfort profile 51-56 is represented to include a series of measured temperatures at times associated with each operation mode. In particular, each thermal-comfort profile 51-56 is commonly illustrated to include a start time (t0) 60, an enter time (t1) 61, a begin-sleeping time (t2) 62, a begin-warming time (t3) 63, and a waking time (t4) 64. Likewise, each thermal-comfort profile 51-56 includes a start temperature (T0) 70, an enter temperature (T1) 71, a begin-sleeping temperature (T2) 72, a minimum temperature (Tmin) 73, and a waking temperature (T4) 74.

The start time 60 represents the beginning of a thermal-comfort profile 51-56, such as a preprogrammed time, a detection of a user's presence, a detection of user intention to go to bed based on set of sensor (e.g., GPS in smartphone signaling user coming home late at night or at other times), or when the user manually turns on or otherwise activates the thermal system. Alternately, the start time 60 may merely indicate the time the system is powered-on. More generally, the start time 60 may indicate when the thermal system actively initiates the thermal-comfort profile (e.g., the thermal system may be turned on 24/7, yet only actively initiate the thermal-comfort profile only when needed/commanded). Thus, the thermal system may be “on”, but if sensors detect user is on vacation, for example, then the thermal-comfort profile may not be initiated.

The enter time 61 represents a time when the user engages the user interface 210, such as the user's entry into bed. Notably, the period of time between start time 60 and enter time 61 may be as short at 10-20 minutes or as long as be indefinite, for example while the thermal system is set to always be prepared to accept a user.

The begin-sleeping time 62 represents the time of a sleep determination (including the time the thermal system initiates actively helping the user falling asleep by lowering the temperature) or the beginning of sleep cycle 50. The begin-warming time 63 represents the time of a final departure from the minimum temperature 73. The waking time 64 represents the time of an awake determination or the end of sleep cycle 50. For purposes of the present disclosure, the sleep cycle 50 may be defined as the period between the begin-sleeping time 62 and the waking time 64. The start temperature 70, enter temperature 71, begin-sleeping temperature 72 and waking temperature 74 represent the targeted measured temperatures at the start time 60, an enter time 61, a begin-sleeping time 62, a begin-warming time 63, and a waking time 64, respectively. The minimum temperature 73 represents the lowest measured temperature of the thermal-comfort profile 51-56.

Notably, the period of time between the begin-warming time 63 and the waking time 64 (“warming period”) may be significantly longer than the period of time between the begin-sleeping time 62 and the begin-warming time 63 (“cooling period”). In particular, the controller may be configured to reach the minimum temperature 73 or the early in the sleep cycle 50, or cool the user at a time associated with an actual or desired circadian rhythm or body clock. For example, the minimum temperature 73 and/or the begin-warming time 63 may be reached during the first third of the sleep cycle 50. Also for example, the cooling period may be predetermined as the first quarter, first 90 minutes, the first 2 hours, or between the first 45 minutes and the first 180 minutes, after an actual or constructive sleep determination. Also for example, the minimum temperature 73 may first be reached at or about a first stage of phase four sleep (deep sleep) (see ref. FIG. 6C). Also for example, the begin-warming time 63 may begin at or about a last (typically second) stage of phase four sleep. Also for example, the minimum temperature 73 may first be reached at or about a time associated with the user's lowest skin temperature, such as 4:30 am. Also for example, the minimum temperature 73 may first be reached at or about a desired time associated with lowest skin temperature, such as 4:30 am local time, where the user has established a biorhythm on another time scale (e.g., jetlagged).

It is understood that sleeping patterns are highly variable from person to person, and may be cut short or interrupted by sleep disruptions. As such, both the cooling and warming periods may themselves vary significantly as well. For example, in some embodiments the cooling period may be on the order of 30 minutes to 3 hours, whereas the warming period may be on the order of 4 to 7 hours. As such, it should be further understood that the common illustration of the various times associated with each operation mode is merely for convenience and clarity of the disclosure.

Qualitatively, each measured temperature of thermal-comfort profiles 51-56 may reflect a temperature of the user, or of one or more points in the sleep environment. In particular, the measured temperatures of the thermal-comfort profiles 51-56 may be measured at one or more points proximate the user, at one or more points in the thermal system, or a combination of both. Selection of the temperature measurement point(s) may also depend on the type of minimum temperature being used.

To illustrate, each thermal-comfort profile 51-56 may reflect a measured temperature at a user interface with the thermal system, a temperature of the coolant in the heat exchanger, a temperature of one or more location within sleep environment, or any combination thereof. For example, each thermal-comfort profile 51-56 may reflect a direct measurement of the user, such as a skin temperature measurement of the user or a temperature measurement of a user interface in thermal contact with the user. Also, for example, each thermal-comfort profile 51-56 may include an indirect measurement, such as a coolant temperature measurement of the heat exchanger or an air temperature measurement near the user. Also, for example, each thermal-comfort profile 51-56 may be calculated or otherwise derived, such as from energy consumption of the system, environmental conditions, and user metrics (e.g. weight, BMI, height, age, sex, etc.).

Quantitatively, the value of the temperatures in each thermal-comfort profile 51-56 will depend on what qualitative measurement is being made. Similar to the sleep determination, the minimum temperature 73 may be may be constructively determined, empirically derived or approximated for a user, or determined substantially in real time. For example, the minimum temperature 73 may be an absolute temperature, where the minimum temperature 73 is a fixed temperature, without reference to another temperature. Also for example, the minimum temperature 73 may be a derivative temperature, where the minimum temperature 73 is determined as a function of another measured temperature (such as an offset or a “delta” from a previously measured temperature). Also for example, the minimum temperature 73 may be a responsive temperature, meaning, the minimum temperature 73 is determined in response to a trigger, such as the user providing feedback that the temperature is too cold. To avoid repeated disruptions, the minimum temperature 73 may be set to an offset of 1-2 degree Celsius before a measured temperature that had previously awaken the user.

Where the minimum temperature 73 is a derivative temperature or an absolute temperature, the measured temperature may be referenced off the user, the thermal system, or a combination of both. For example, one combination of both may include taking the first of a derivative and an absolute temperature that breaches a threshold temperature, and treat that temperature as the minimum temperature 73.

In contrast, where the minimum temperature 73 is a responsive temperature, the minimum temperature 73 may be merely identified in response to input or feedback from the user. For example, a minimum temperature 73 may be set based, at least in part, on user commands to the controller to increase or decrease temperature. Also for example, a minimum temperature 73 may be set where sensor feedback from the user indicates the temperature is too low, such as a detection of vasoconstriction (narrowing of the blood vessels leading to the skin capillaries) or changes in heart beating and breathing patterns.

As discussed above, the controller may operate the heat exchanger according to a plurality of operation modes, which may extend over and outside a sleep cycle 50 of a user. In particular, the controller 500 is may be configured to operate the heat exchanger 200 according to a sleeping mode 593 and a warming mode 594 over the course of the sleep cycle 50. The controller may be further configured to operate the heat exchanger according to a pre-enter mode 591 and/or a pre-sleeping mode 592 prior to the sleep cycle 50. In addition the controller may be further configured to operate the heat exchanger according to an overheat mode 595 and/or a manual mode 596 independent of or in conjunction with the sleep cycle 50.

For reference, where one or more of the listed operation modes is not included in one of the illustrated thermal-comfort profiles 51-56, one or more of the above-referenced times and temperatures may still be included for consistency with other figures. For example, as discussed below, FIG. 6A refers to start time 60 and enter time 61, as well as start temperature 70 and enter temperature 71, however, in some embodiments, the controller might not actively operate the heat exchanger until the sleeping mode 593 at the begin-sleeping time 62. Thus, the prior times and temperatures may merely refer to local ambient conditions.

Referring to FIG. 6A, the thermal-comfort profiles 51, 52 include or represent the sleeping mode 593 and the warming mode 594, but omit a pre-enter mode and a pre-sleeping mode. In particular, from the start time 60 and through the begin-sleeping time 62, the thermal system may passively remain at an ambient temperature without operation of the heat exchanger. The controller begins to operate the heat exchanger at the begin-sleeping time 62 in the sleeping mode 593. This is followed by the warming mode 594 between the begin-warming time 63 and the waking time 64. Notably, the heat exchanger is operated as a passive device, then as a cooler, and then as a heater.

The thermal-comfort profile 51 is shown here as series of interconnected linear sections. At initiation, or start time 60, the thermal system passively remains constant at an ambient temperature until the begin-sleeping time 62. In particular, the start temperature 70, the enter temperature 71 and the begin-sleeping temperature 72 are generally constant at the ambient temperature. Next, for the sleeping mode 593, the thermal-comfort profile 51 decreases linearly to the minimum temperature 73, and remains constant at the minimum temperature 73 until the begin-warming time 63. Next, for the warming mode 594, the thermal-comfort profile 51 increases linearly from the minimum temperature 73 at the begin-warming time 63 to the waking temperature 74 at the waking time 64. Notably, the slope of the thermal-comfort profile 51 approaching the minimum temperature 73 is generally steeper than the slope of the thermal-comfort profile 51 departing the minimum temperature 73.

According to one embodiment, the thermal-comfort profile of the thermal system may be non-linear. In particular, and as illustrated, thermal-comfort profile 52 is a curve substantially tracking or fitted to the linear thermal-comfort profile 51. It is understood that, while the temperature between start time 60 and begin-sleeping time 62 is conveniently illustrated as a constant value, the actual value may vary as a result of the user's body heat and/or changing ambient conditions (e.g., evening cooling). For example, thermal-comfort profile 52 includes a representation of a transient temperature change due to the user entering the bed (preceding begin-sleeping time 62), where the user adds a nominal amount of heat to the thermal system.

As discussed above, at start time 60 and enter time 61, the controller does not actively operate the heat exchanger in this illustrated embodiment. Rather, the controller begins to actively operate the heat exchanger at the begin-sleeping time 62. As such, here, a pre-enter mode and/or a pre-sleeping mode may be disregarded or merely made by designation, as the temperature of the thermal system is generally the same as the ambient temperature. For example, a “designated pre-enter mode” may start at an activation time, such as turning on the thermal system or a detection of proximity to the thermal system, without any operation of the heat exchanger. Also for example, a “designated pre-sleeping mode” may start when the user enters the bed, but without any operation of the heat exchanger. The designated pre-sleeping mode then continues until a sleep determination. This may be advantageous where a sleep determination is not actually made. Notwithstanding, in other embodiments, temperatures 70-73 may be actively controlled by the controller. For example, in winter, the start temperature 70 may be warmer than room temperature, such as an increase of 1-2 degrees Celsius or more by enter time 61. Also for example, in summer, the start temperature may be lower than room temperature, such as a decrease 1-2 degrees Celsius or more by enter time 61.

The sleeping mode 593 includes conductively cooling the user along a thermal-comfort profile 51, 52 to a minimum temperature 73. In particular, the thermal-comfort profile 51, 52 and the minimum temperature 73 may approach, and remain just above a threshold defined as disruptive to sleeping, as discussed further below. This threshold may be estimated or otherwise predetermined for a general user or for a particular user. In addition, this threshold may be calculated, determined in response to sensor feedback, determined in response to user feedback, or any combination thereof. Beneficially, an improved sleep may be experienced by aggressively cooling the user during the sleeping mode 593 to just above a threshold defined as disruptive to sleeping.

In some embodiments, the minimum temperature 73 may be reached at the begin-warming time 63. However, in other embodiments, as illustrated by thermal-comfort profile 51, where the minimum temperature 73 is reached prior to the begin-warming time 63, the minimum temperature 73 may be maintained or held constant until begin-warming time 63. Similarly, in alternate embodiments (see ref., FIG. 6C), where the minimum temperature 73 is reached prior to the begin-warming time 63, the measured temperature may be raised and returned to the minimum temperature 73 until the begin-warming time 63.

According to one embodiment, the sleeping mode 593 may aggressively cool the user by a predetermined amount. In particular, while in the sleeping mode 593, the measured temperature may lowered by at least 5 degrees Celsius from a begin-sleeping temperature 72. For example, where a coolant temperature is measured at the user interface, the controller may operate the heat exchanger to remove heat from the coolant until at least a 5 degrees Celsius drop is measured at the user interface. Similarly, where a coolant temperature is measured in the heat pump module (e.g., upstream of the reservoir 252), the controller may operate the heat exchanger to remove heat from the coolant until at least a 5 degrees Celsius drop is measured. Also for example, the controller may operate the heat exchanger in the sleeping mode 593 to reach a minimum temperature 73 of at least 10 degrees Celsius below begin-sleeping temperature 72. Also for example, the controller may operate the heat exchanger in the sleeping mode 593 to reach a minimum temperature 73 of 5 degrees Celsius to 10 degrees Celsius below begin-sleeping temperature 72, and/or to maintain a temperature drop of 5 degrees Celsius to 10 degrees Celsius from begin-sleeping temperature 72. Also for example, the controller may operate the heat exchanger in the sleeping mode 593 to reach a minimum temperature 73 of 5 degrees Celsius to 15 degrees Celsius below begin-sleeping temperature 72, and/or to maintain a temperature drop of 5 degrees Celsius to 15 degrees Celsius from begin-sleeping temperature 72. Also for example, the controller may operate the heat exchanger in the sleeping mode 593 to reach a minimum temperature 73 of 10 degrees Celsius to 15 degrees Celsius below begin-sleeping temperature 72, and/or to maintain a temperature drop of 10 degrees Celsius to 15 degrees Celsius from begin-sleeping temperature 72. Also for example, the controller may operate the heat exchanger in the sleeping mode 593 to reach a minimum temperature 73 of 15 degrees Celsius.

The sleeping mode 593 is initiated at begin-sleeping time 62 by a sleep determination as discussed above. In particular, the sleep determination may be determined via sensor input, which is representative of a sleep state. For example, a sleep determination may be made with measured sensor data correlated to a sleep state, such as pulse, brainwave, motion, thermal equilibrium data, etc. Alternately, the sleep determination may be based on an expected time of sleep. For example, the controller or the user may set a time where sleep is anticipated (e.g., 30 minutes after a triggering event). The triggering event may be the user's entry into bed, a detection of a user's presence, a manual triggering by the user, etc. Thus, in some instances, the sleep determination may not require the user to actually be asleep or the user may actually be asleep prior to the sleep determination. Similarly, the sleep mode 593 may be initiated by the controller without any requirement for user to be asleep to help user fall asleep.

The warming mode 594 includes conductively warming the user along the thermal-comfort profile 51, 52 from the minimum temperature 73 at the begin-warming time 63 toward the waking temperature 74 at the waking time 64. In contrast to the sleeping mode 593, the warming mode portion of the thermal-comfort profile 51, 52 may be gentler, or have aggregate or average slope that is substantially less steep. In particular, the thermal-comfort profile 51, 52 may gradually increase from the minimum temperature 73 to the waking temperature 74 over the entire warming period, between the begin-warming time 63 and the waking time 64. For example, the warming mode portion of the thermal-comfort profile 51, 52 may linearly increase from the minimum temperature 73 to the waking temperature 74. Also for example, the warming mode portion of the thermal-comfort profile 51, 52 may include departures from a generally linear path while minimizing an overall or aggregate departure from the generally linear path. Moreover, since the warming period may be significantly longer than the cooling period the slope of the warming mode portion of the thermal-comfort profile 51, 52 may be further flattened and gradual in reaching the waking temperature 74.

Generally, the waking temperature 74 may be set to a temperature that is comfortable to the user and/or aids in waking up. For example, the waking temperature 74 may be set to the ambient temperature at the waking time 64. Also for example, the waking temperature 74 may be set above the ambient temperature. This may be beneficial where the ambient temperature is subjectively cool or as an aid to encourage getting out of bed. Also for example, the waking temperature 74 may be set below the ambient temperature. This may be beneficial where the ambient temperature is subjectively warm or where it is desirable to extend the user's sleep period. Furthermore, the waking temperature 74 may be predetermined based on weather conditions, user feedback, user preferences, and other considerations.

In an alternate embodiment, the thermal-comfort profile may include an aggressive warming up period, for example, in case the user needs to be awaken in a short period of time. As such, the period between begin-warming time 63 and wake up time 64 could be as short as 10 minutes. Accordingly, the waking temperature 74 may be set approximately 5-10 degrees Celsius higher than begin-sleeping temperature 72.

Referring to FIG. 6B, the thermal-comfort profile 53 is superimposed onto the thermal-comfort profile 51 of FIG. 6A for reference. Here, the thermal-comfort profile 53 illustrates modifications or adjustments to a baseline, which are responsive to user feedback. In particular, adjustments to the previous thermal-comfort profile 51 result in the illustrated thermal-comfort profile 53. It should be understood that the thermal-comfort profile 51 is selected by way of example, and that other the thermal-comfort profiles may be modified. Likewise, the controller may increase or decrease the heating/cooling above or below the illustrated thermal-comfort profile 53. In addition, generic event times 81-85, are included for clarity in illustrating the embodiments, but are not limiting in any way.

As above, the thermal-comfort profile 53 is shown here as series of interconnected linear sections (which in other embodiments may be non-linear). At initiation, or start time 60, the thermal system may passively remain constant at an ambient temperature through the enter time 61. In particular, the start temperature 70 and the enter temperature 71 are shown generally constant at the ambient temperature. Next, for the pre-sleeping mode 592, the thermal-comfort profile 53 may passively remain constant at the ambient temperature until a first event time event time 81, where it increases until the begin-sleeping time 62. Next, for the sleeping mode 593, the thermal-comfort profile 53 decreases linearly until a second event time 82, where it remains constant for a thermal rest period 80, and then resumes decreasing linearly to the minimum temperature 73 and remains constant at the minimum temperature 73 until the begin-warming time 63.

Next, for the warming mode 594, the thermal-comfort profile 53 increases linearly from the minimum temperature 73 at the begin-warming time 63 until a third event time 83, where the thermal system continues to heat the user, but at a more gradual rate; subsequently, at a fourth event time 84, the thermal-comfort profile 53 is held constant until a fifth event time 85, where it resumes increasing linearly to the waking temperature 74 at the waking time 64.

As above, here, the controller does not actively operate the heat exchanger between start time 60 and enter time 61. Also as above, a pre-enter mode may be disregarded or merely made by designation. However, in the illustrated embodiment, the controller may be further configured to operate the heat exchanger according to pre-sleeping mode 592. As such, thermal system may passively remain at an ambient temperature without operation of the heat exchanger until the enter time 61, at which point the controller may actively operate the heat exchanger.

The pre-sleeping mode 592 is directed toward providing subjective thermal comfort to the user upon interfacing with the user interface (e.g., entry into bed) and until falling asleep or the start of the sleep cycle 50. In particular, the pre-sleeping mode 592 may include, conductively warming the user, conductively cooling the user, holding a constant temperature, or any combination thereof, between the enter time 61 and the begin-sleeping time 62. The enter time 61 may be a predetermined time from the start time 60 of the thermal system (e.g., 5 minutes), or may be determined by the presence of the user in contact with the user interface. By making the user more comfortable, the user may fall asleep faster, among other benefits. However, it is understood that subjective thermal comfort, by definition, is highly variable, and moreover, is typically unique to the user at the given moment.

According to one embodiment, subjective thermal comfort may be estimated or otherwise predetermined. In particular, the pre-sleeping mode 592 may maintain the user interface at an estimated comfortable temperature for a general user. For example, the pre-sleeping mode 592 may set the bed temperature to 33 degrees Celsius from the time the user enters the bed until the user falls asleep. Also for example, the enter temperature 71 may be set below ambient temperature to begin cooling the user and help the user fall asleep.

Alternately, the pre-sleeping mode 592 may maintain the user interface at a preset estimated comfortable temperature for a particular user. For example, a particular user may select or otherwise set a begin-sleeping temperature 72 for the period between the enter time 61 and the begin-sleeping time 62 preset, which may result in warming, cooling, or maintaining user interface temperature. Also for example, a particular user whose subjective thermal comfort is perceived as being cold (i.e., “feels” hot and wants to cool down), the enter temperature 71 and the begin-sleeping temperature 72 may be equal or similar to the minimum temperature 73. Moreover, this may be predetermined for a user based on body mass index (BMI) such as a BMI>25.

According to one embodiment, the pre-sleeping mode 592 may further include receiving and responding to a user input or automated data. In particular, the subjective thermal comfort may be estimated in advance, and subsequently modified by feedback from user input or automated data.

The user input may be provided by any convenient means. For example, the user control may include a “heat” and/or “cool” button, configured such that the user may directly provide input to the controller indicating when the current temperature is subjectively too cool or too warm, respectively. Also for example, the controller and one or more user sensors may be configured to detect specific user motions, such as a single or multi tap on the mattress, the user interface, or heat exchanger, or such as hand swiping at specific location (e.g., top or side of mattress).

To illustrate, here, the pre-sleeping mode 592 portion of the thermal-comfort profile 53 begins at an ambient temperature at the enter time 61 and continues until a determination at event time 81 that the sleep environment is too cool. The determination may be made by any means, such as feedback from user input or automated data. For example, the user may press heat button or area of the user control. Upon this determination, the controller begins to heat the sleep environment by a first increment (e.g., an increase of 1 degree Celsius) until the begin-sleeping time 62. It is understood that the thermal-comfort profile 53 may be further modified in response to subsequent feedback or reaching a predetermined limit or based on past user data or based on other users historical data, for example, using adaptive learning techniques.

Similarly, automated data may be provided by any convenient means, which may be interpreted as requiring heating, cooling, or no temperature change. For example, the automated data may include feedback from user sensors indicative of subjective thermal comfort (e.g., sensors for skin temperature, body position, core-to-extremity temperature differential, sweat/humidity, heart rate, breathing, etc.), environment sensors (e.g., sensors for outdoor temperature, indoor temperature, etc.), or any otherwise available data (historical user data, calendar/season data, weather data, etc.).

To illustrate, according to one embodiment, the user may be detected as having an increased breathing rate and heart rate. This may then be interpreted as indicating a non-optimal thermal-comfort profile. In response, the controller 500 may then warm the user. If no further user feedback is received, or if the sensor data is otherwise back to normal, this new “corrective response” information may be stored for future use. Conversely, if user feedback is received or if sensor data otherwise indicates an increasing deviation from the norm, the controller 500 may cool the user. In addition, the controller 500 may be further configured to use this sequence (and/or similar techniques) to adaptively learn to interpret sensor data and/or create actual thermal-comfort profiles for specific users.

As above, the sleeping mode 593 is directed toward aggressively cooling the user upon falling asleep or the start of the sleep cycle 50, without disrupting sleep. However, the sleeping mode 593 may further include receiving and responding to a user input or automated data. In particular, the “aggressive cooling” of the thermal-comfort profile 53 may be estimated or predetermined, and subsequently modified. For example, here, a determination is made at the second event time 82 that the sleep environment is again too cool for the user. The determination may be made by feedback from user input or automated data similar to the pre-sleeping mode 592. For example, the user may momentarily wake, and press a heat button or perform some predetermined motion that is detected by user sensor 400. Also for example, the determination may be an automated determination, such as user sensors detecting that the user has assumed a fetal position.

Responsive to the determination, the controller may modify the operation of the thermal system. Here, the controller stops cooling the user and holds the temperature constant for the thermal rest period 80 (e.g. 5 minutes), after which, the controller may resume aggressively cooling the user at the previous rate. In other embodiments, the thermal rest period 80 may be for a greater or lesser duration, or may instead include heating the user (e.g., where the user makes repeated button presses, or where a pulse-ox sensor indication of detection of vasoconstriction). Similarly, where a determination is made that the sleep environment is too warm for the user (i.e., not cooling fast enough), the controller may increase cooling.

According to one embodiment, the controller may provide a tempered response to the determination. In particular, the controller may continue to cool the user, but at a less “aggressive” rate (e.g., half rate), in response to the determination at the second event time 82. Alternately, the controller may continue to cool the user at the same rate, but increase feedback data resolution. For example, the controller may increase a sampling rate, increase the number of sensors reporting, or cross-reference with other types of sensor feedback, to name a few. This may be beneficial where the where the determination is based on more subtle or inconclusive feedback, such as an increase in breathing, heart rate, or position changes, for example.

By receiving and using the feedback from user input or the automated data, the controller may also modify operation parameters, such as lower the minimum temperature 73 or drive the thermal-comfort profile 53 closer to a threshold disruptive to sleeping. In particular, the thermal-comfort profile 53 may generated or modified by the controller, responsive to feedback from the user, feedback from a plurality of users, learning algorithms, and other determinations which may be made. For example, subsequent iterations of the thermal-comfort profile 53 (e.g., the next evening) may be modified or “learn” from the feedback.

As with determining subjective thermal comfort, a threshold disruptive to sleeping may be highly variable and unique to both the user and the given sleep cycle 50. As such, the user input or the automated data may be used to guide the controller and modify the thermal-comfort profile 53 (or even guide the controller's operation of the heat exchanger without a predetermined or otherwise recorded thermal comfort profile) substantially in real time (i.e., during operation or during the sleep cycle 50). For example, the user input or the automated data may indicate that the operation of the thermal system is too cool, too warm, too aggressive, or too gentle, requiring modification or additional feedback. Automated feedback may be beneficial prior to, during, and subsequent to a disruption in the controller's determining and modifying heat exchanger operation or gathering more data. Direct user input, however, is indicative of a disruption and may be beneficial in modifying heat exchanger operation more assertively and both immediately in the future.

Moreover, the controller may actively “test” and modify the thermal-comfort profile 53. In particular, the controller may operate the heat exchanger in an increasingly aggressive manner until a user response is received or disruption is otherwise detected. For example, during the sleeping mode 593, the controller may cool the user at a first rate, and incrementally increase the rate until a sleep disruption is identified, such as detecting a heat button depression, or until a limiting threshold is reached (e.g., reflecting a reasonable maximum tolerance or limit to human cooling). This may be particularly advantageous during a “learning” stage when the thermal system is initially operated by the user and no historical data is recorded.

According to one embodiment, the limiting threshold may be set to detect changes in user tolerance or system creep. In particular, rather than setting the limiting threshold to an absolute value such as a maximum user tolerance, the limiting threshold may be set to a relative value, such as a ratio or percent change from an existing thermal-comfort profile. For example, the limiting threshold may be set to a multiplier of the cooling rate of the thermal-comfort profile 53 (e.g., two times the slope). Also for example, the limiting threshold may be set to a range (e.g., 0.5 to 5 times) of the cooling rate of the thermal-comfort profile 53. Other modifications, particularly with narrower tolerances, are contemplated, as this embodiment may be beneficial in ongoing operations and cycles of the thermal system or subsequent to a baseline user tolerance being determined. Furthermore, this and other adaptive learning aspects of the disclosure may be linked to or otherwise associated with a particular user, as users of the thermal system may vary.

As above, the warming mode 594 is directed toward gradually warming the user toward the waking temperature 74 at the waking time 64. Here, however, a determination is made at the third event time 83 that the sleep environment is too warm for the user. As above, the determination may be made by feedback from user input or automated data. For example, one or more user position sensors may indicate the user's extremities are extended, or otherwise assumed a sprawled out sleeping position. Here, responsive to the determination, the controller continues to warm the user, but at an even “gentler” rate. As above, this may be beneficial where the where the determination is based on more subtle or inconclusive feedback. Notwithstanding, however, another determination is made at the fourth event time 84 that the sleep environment is still too warm for the user. This time however, the controller may stop warming the user, and hold the temperature constant. In other embodiments, the response may include cooling the user. These stronger responses may be beneficial where the where the determination is based on a sleep disruption or conclusive feedback. At the fifth event time 85, another determination is made, now however, that the sleep environment is too cool for the user. As above, the determination may be based on may be made by feedback from user input, automated data, or other factors. Here, the controller may gradually increase the temperature to the waking temperature 74 over the remaining warming period, until the waking time 64. Moreover, the waking temperature 74 and/or the waking time 64 may be modified during operation of the thermal system, for example, based on the one or more responsive determinations.

Referring to FIG. 6C, the thermal-comfort profile 54 is shown with a series of five exemplary sleep sub-cycles 40-44. Each sleep sub-cycle 40-44 is illustrated as a step-curve, wherein each step represents a sleep stage. Here, the thermal-comfort profile 54 illustrates modifications or adaptions of thermal-comfort profile 54 responsive to both determinations of sleep stage and to user feedback. As with the exemplary thermal-comfort profiles, it should be understood that the five exemplary sleep sub-cycles 40-44 are selected by way of example, and that sleep sub-cycles may vary drastically from user-to-user and from sleep cycle-to-sleep cycle. In addition, generic event times 81-86, are included for clarity in illustrating the embodiments, but are not limiting in any way, and unlike other reference numbers, are generally unrelated to the event times of FIG. 6C.

With regard to the sleep sub-cycles 40-44, sleep is commonly divided into two broad types, rapid eye movement sleep (REM) and non-rapid eye movement (NREM) sleep. During sleep, the body cycles between non-REM and REM sleep. REM is treated as one phase or “stage”, and NREM is further divided into four stages, which correspond to the depth of sleep. For example, Stage one and Stage two are considered “light sleep”, and Stage three and Stage four are considered “deep sleep” or slow-wave sleep (SWS). In deep sleep the sleeper is less responsive to the environment, and many environmental stimuli no longer produce any reactions. While some sleep models consolidate Stage three and Stage four, discussion directed toward these distinctions are beyond the scope of the present disclosure.

Each sleep stage has a distinct set of associated physiological, neurological and psychological features, which are known in the art and beyond the scope of this disclosure. As illustrated, from the “awake” state 35, the sleep sub-cycles 40-44 generally follow the downward phase order of: REM 45, Stage one 46, Stage two 47, Stage three 48, and Stage four 49, and an upward phase order of: Stage four 49, Stage three 48, Stage two 47, Stage one 46, and REM 45. However, over the course of the entire sleep cycle 50, individual sleep sub-cycles may be shallower, and not reach the full Stage four sleep. In particular, there are typically a greater amount of Stages three and four 48, 49 early in evening or sleep cycle 50 and longer REM later in the sleep cycle 50 (e.g., towards the morning). In humans, a sleep sub-cycle 40-44 lasts on average 90 to 110 minutes. By taking advantage of particular sleep states where user sensitivity is diminished, the thermal-comfort profile 54 may be accelerated and the user may be more aggressively cooled.

Regarding the thermal-comfort profile 54 of the controller, as above, the thermal-comfort profile 54 is illustrated as series of interconnected segments, which include the pre-sleeping mode 592, the sleeping mode 593, and the warming mode 594. Also as above, the thermal-comfort profile 54 remains at an ambient temperature from the start time 60 until the enter time 61, at which point the controller enters the pre-sleeping mode 592. In the pre-sleeping mode 592 the thermal-comfort profile 54 initially remains at the ambient temperature, but at a first event time 81 the thermal-comfort profile 54 begins to gently decrease until the begin-sleeping time 62.

Next, in the sleeping mode 593, the thermal-comfort profile 54 decreases linearly until a second event time 82, where it decreases more aggressively to the minimum temperature 73 and briefly remains constant until a third event time 83. At the third event time 83, the thermal-comfort profile 54 increases slightly for a thermal rest period 80 and returns to the minimum temperature 73. At a fourth event time 84, corresponding to an upward transition between Stage four 49 and Stage three 48, the thermal-comfort profile 54 increases even more slightly. At a fifth event time 85, corresponding to a downward transition between Stage three 48 and Stage four 49 the thermal-comfort profile 54 returns to the minimum temperature 73, and remains constant.

Next, in the warming mode 594, the thermal-comfort profile 54 increases linearly from the minimum temperature 73 at the begin-warming time 63 toward the waking temperature 74. Notably, here, the begin-warming time 63 corresponds to the last upward transition between Stage four 49 and Stage three 48. At a sixth event time 86, the thermal-comfort profile 54 begins to increase more rapidly, and continues linearly at the increased rate until reaching the waking temperature 74, where it remains constant until the waking time 64.

Operationally, the thermal system may passively remain at an ambient temperature without operation of the heat exchanger until the enter time 61, at which point the controller enters the pre-sleeping mode 592.

As above, the pre-sleeping mode 592 is directed toward providing subjective thermal comfort to the user. Here, is the subjective thermal comfort is estimated, and the controller sets the bed temperature to a comfortable temperature, which conveniently may or may not be the ambient temperature. At the first event time 81, however, the controller begins to actively operate the heat exchanger. For example, the controller may correlate sensor data of an elevated heart rate with recent physical activity data recorded on a wearable device of the user to determine that the user would be subjectively more comfortable with a bed temperature slightly below an otherwise comfortable temperature. This data may be further correlated with historical feedback from the user in support of the determination. In response, the controller may then operate the heat exchanger to lower the sleep environment or bed temperature.

As above, the sleeping mode 593 is directed toward aggressively cooling the user at the start of the sleep cycle 50, without disrupting sleep. Here, a predetermined “aggressive” cooling rate may be taken at the begin-sleeping time 62. At the second event time 82, a determination is made that the user has entered Stage three 48 sleep, and in response, the controller may cool the user at the highest rate consistent with limitations of or placed on the thermal system. The cooling may continue until reaching the minimum temperature 73, and briefly remain constant.

Shortly thereafter, the third event time 83 is reached, indicating a sleep disruption has occurred or is likely to occur (e.g., a detection of vasoconstriction, heat button press, excessive motion, fetal position, etc.). In response to this determination, the controller may briefly warm (e.g., 1 degree Celsius) the user for a thermal rest period 80. The thermal rest period 80 may last for a predetermined amount of time, or may be responsive to feedback data indicating the disruption has been mitigated. After the thermal rest period 80, controller may return the thermal-comfort profile 54 to the minimum temperature 73.

At the fourth event time 84, a determination is made that the user has left Stage four sleep 49 and entered Stage three 48 sleep. In response, the controller may gently warm (e.g., 0.5 degree Celsius) the user until reaching the fifth event time 85, where it is determined the user has returned to deep sleep. The user's sleep state can be detected by user sensor 400, for example, configured as a brain-wave recorder (e.g., EEG). Likewise the user's sleep state can be detected by a change in multiple variable such as breathing rate, heart rate, heart rate variability, etc. In response to the determination at the fifth event time 85, controller may return the thermal system to the minimum temperature 73 and remain there until the begin-warming time 63.

As above, the warming mode 594 is directed toward gradually warming the user toward the waking temperature 74 at the waking time 64. Here, the begin-warming time 63 may correspond to the last upward transition between Stage four 49 and Stage three 48. In particular, the controller may be configured to set the begin-warming time 63 at or about the end of the last sleep sub-cycle to include deep sleep. For example, since here there are only two sleep sub-cycles 40, 41 that reach deep sleep, the second sleep sub-cycle 41 is the last sleep sub-cycle to include deep sleep. In other embodiments, the last sleep sub-cycle to include deep sleep may be determined based on a variety of criteria including, but not limited to historical data of the user, a number of hours set as the sleep cycle 50, retrospectively (i.e., presuming a current deep sleep stage is the last until detecting a subsequent deep sleep stage). At the begin-warming time 63, the controller may warm the user linearly from the minimum temperature 73 toward the waking temperature 74 at the waking time 64.

At the sixth event time 86, a determination is made that the sleep environment is too cool for the user. As above the determination may be made via user input or automated data. For example, user sensor 400 can detect the user in a fetal position indicating a possible too cold environment. Alternatively, changes in breathing rate, heart rate, or other user sensors might provide additional information. For example, for a specific user, an increase of heart rate or of heart rate variability might indicate an imminent arousal, which may be responded to by the controller 500 to warm the user in light of the current time, location, and other variables . . . . In response, the controller may increase the warming rate. For example, the controller may increase the warming rate between 25 and 100 percent, and continue until reaching the waking temperature 74. In other embodiments, the controller may raise or lower the waking temperature 74 as well, for example, based on automated data, environment sensors, or any otherwise available data.

Referring to FIG. 6D, the thermal-comfort profiles 55, 56 include the pre-sleeping mode 592 the sleeping mode 593 and the warming mode 594, substantially as described above. However, both thermal-comfort profiles 55, 56 also include a pre-enter mode 591. Note, in this figure, a suffix “A” is applied to certain reference numbers merely for clarity, designating an association with latter thermal-comfort profile 56.

Additionally, thermal-comfort profile 55 includes an overheat mode 595 and a manual mode 596. As such, the thermal-comfort profile 55 also illustrates an exit temperature (T5) 75 corresponding to an exit time (t5) 65, a begin-overheat temperature (T5) 76 corresponding to a begin-overheat time (t5) 66, and an end-overheat temperature (T5) 77 corresponding to an end-overheat time (t5) 67.

The pre-enter mode 591 is directed toward preconditioning the sleep environment. In particular, the user, the controller, or a combination thereof may set a desired state for the sleep environment, to which the controller may operate the heat exchanger prior to the user's entry into the sleep environment (at enter time 61). For example, the heat exchanger may pre-warm or pre-cool a bed to a before the user enters it.

The pre-enter mode 591 may include receiving an initiation command, and operating the heat exchanger to precondition the sleep environment to the enter temperature 71, 71A, preferably at or prior to enter time 61. As above, the enter time 61 may be a predetermined time from the start time 60, or determined by the presence of the user in contact with the user interface. The initiation command may be provided the start time 60, for example, manually by the user or automatically (e.g., responsive to a clock time, a proximity determination of the user, etc.).

Furthermore, the enter temperature 71, 71A may be any subjectively comfortable temperature, for example as determined by the user, as determined above for subjective comfort, or the like. According to one embodiment, the pre-enter mode may provide for substantially the same subjective thermal comfort conditions of the pre-sleeping mode 592 prior to the user getting into bed. For example, the thermal-comfort profile 55 may represent a condition where the sleep environment begins cool and requires warming, such as wintertime, or where the user is subjectively cool (e.g., after a cool shower or swim). Also for example, the thermal-comfort profile 56 may represent a condition where the sleep environment begins warm and requires cooling, such as summertime, or where the user is subjectively warm (e.g., after a warm shower or exercise). In both the thermal-comfort profiles 55, 56, the controller may operate the heat exchanger to bring the user interface to a predetermined enter temperature 71, 71A, respectively, at enter time 61.

The manual mode 596 is directed toward providing the user with control over the operation of the heat exchanger. In particular, the user may set a desired temperature for the heat exchanger to follow. In addition, the user may set said operation for a period of time. For example, the user may directly operate the heat exchanger in conjunction with the thermal-comfort profile 55, where the user is warmed (or cooled) between the waking time 64 and an exit time (t5) 65 toward an exit temperature 75. The exit temperature 75 may be predetermined or made in response to user input or other feedback. Also for example, the user may operate the heat exchanger independent of any thermal-comfort profile, merely as a heater or cooler. Additionally, the user may override a controller operation of the heat exchanger. For example, the user may wake during the sleeping mode 593, and, rather than provide a user input to provide heat or a thermal rest (e.g., interrupt cooling for an intermittent period), the user may override the sleeping mode 593 and set thermal system to a predetermined temperature for a predetermined period or until initiation of a subsequent mode of the thermal system.

The overheat mode 595 is directed toward improving the sleep environment by heating it to levels well above thermal comfort levels for a period of time. In particular, the controller may heat the sleep environment (e.g., via the user interface) to an overheat temperature (Tmax) 76 between a begin-overheat time 66 and an end-overheat time 67.

The overheat temperature 76 and the period between the begin-overheat time 66 and the end-overheat time 67 may be selected based on whether the overheat mode 595 is being used for removing moisture, pasteurizing, sterilizing, or otherwise disinfecting the sleep environment. Moreover, their combination may be selected based on an anticipated operation time. For example, the overheat temperature 76 may be set approximately at 40 degrees Celsius, above 50 degrees Celsius, above 70 degrees Celsius, approximately at 85 degrees Celsius, between 50 degrees Celsius and 70 degrees Celsius, between 70 degrees Celsius and 90 degrees Celsius, between 40 degrees Celsius and 130 degrees Celsius (with a coolant such as liquid silicone, which can sustain such high temperatures), at maximum operational temperature of the thermal system, or other conventional temperatures for the above uses. Also for example, the period between the begin-overheat time 66 and the end-overheat time 67 may be on the order of 30 minutes to 1 hour, several hours (e.g., 4-10 hours), or for extended periods (e.g., 12-24 hours).

According to one embodiment, the overheat mode 595 may also be directed toward maintenance or performance of the thermal system. In particular, the controller may operate the heat exchanger according to the overheat mode 595 to reduce liquid fouling inside the heat exchanger and other parts of the thermal system in contact with fluid or coolant. In this scenario, the overheat temperature 76 may be set above 50 degrees Celsius for at least one hour. In another embodiment the overheat temperature 76 may be set above 50 degrees Celsius for a longer time (e.g., 2 hours) to pasteurize the heat exchanger, as well as the mattress and/or sleep environment close to the heat exchanger.

This may be particularly useful in a multi-user scenario like hotels, cruises, or other applications where different users might use the thermal system at different times. For example, the overheat mode 595 may be automated to initiate a predefined time after the waking-time 64, can be remotely controlled, or can be initiated by the user. In addition, the overheat mode 595 may be stopped anytime a user sensor detects a user in close proximity to the heat exchanger. In this particular case, the controller may activate a fast cooling sequence to rapidly bring the coolant temperature within normal range (e.g., less than 37 degrees Celsius), activate an alarm, or send an alert for the potential danger.

Although particular aspects are described herein, many variations and permutations of these aspects fall within the scope of the disclosure. Although some benefits and advantages of the preferred aspects are mentioned, the scope of the disclosure is not intended to be limited to particular benefits, uses, or objectives. Rather, aspects of the disclosure are intended to be broadly applicable to different user applications, sleep environments, industrial applications, and operation profiles, some of which are illustrated by way of example in the figures and in the foregoing description of the preferred aspects. The detailed description and drawings are merely illustrative of the disclosure rather than limiting, the scope of the invention being defined by the appended claims and equivalents thereof.

### INDUSTRIAL APPLICABILITY

The present disclosure generally pertains to a thermal system for conditioning the sleep environment and managing skin temperature of a user during sleep, and is applicable to personal use, commercial use, professional use, research use, etc. The thermal system and method embodiments described herein may be suited for any number of industrial applications, such as, but not limited to, various aspects of the hospitality industry (e.g., hotels, cruise ships, and other fields where a sleep environment is provided), retail industry, aerospace and transportation industry (where a sleep environment is provided), military industry or any other industry where improved human performance is desired, to name a few examples.

Furthermore, the described embodiments are not limited to use in conjunction with a particular type of sleep environment. There are numerous thermal system configurations and use environments that are applicable here. In particular, within each industry and use case, there may be many variations, depending on the application, use, and/or performance desired. For example, when configured to include a pad or mat placed on a bed (e.g., as the user interface), it may vary in size, such as a standard bed size, or may be configured to join with another mat or pad, such that they are operable in conjunction with each other as a single user interface. Moreover, in said example, the coupled user interfaces may operate independently, so as to provide an independent sleeping environment for a plurality of simultaneous users.

Also for example, while the user interface of the heat exchanger is illustrated above as including a liquid thermal circuit, other types of heat exchanger media may be used, including but not limited to gas (e.g., compressed), gel, phase transition material, or solid (e.g., passive heat sink, distributed thermoelectric heat pump (e.g., reversible Peltier device distributed across the user interface). In addition, aspects of the system may be used in conjunction with or integrated into another existing thermal system, such as home HVAC, aircraft environmental control system, any controller or mobile device having a processor configurable (e.g., via software download) to communicate with and issue appropriate commands to the heat exchanger.

Generally, embodiments of the presently disclosed thermal system are applicable to personal use, entertainment, user experience, preventative care, research, and innovation of sleep environments. The disclosed teachings may lead to improved sleep, rest, and human performance, disrupt existing sleeping patterns, or lead to decreased involuntary sleep events and/or susceptibility to disruptions. In addition, embodiments of the presently disclosed thermal system may be applicable new use applications, retrofitting an existing sleep environment, and to testing or research, wherein the sleep environment is dynamically modified.

While there are many different benefits, not all of which are specifically identified herein, one or more aspects of the present invention may include one or more of the following advantages. The thermal system and method may provide for better rest for people with insufficient sleep, improved oxygenation for sleep apnea patients, more restful sleep, less time falling asleep, longer sleep cycle, and improved shifting of circadian rhythm (e.g., for mitigation and/or prevention of jet-lag). Advantageously, the thermal system and method is generally non-intrusive, and embodiments have the added benefit of not requiring a user to wear a device while trying to sleep. Moreover, the thermal system and related methods are adaptable, and can be implemented in a user's home, in a hotel, or even professionally, such as in a clinical or hospital setting. The thermal system may operate without requiring expertise or training, and may also collect, correlate, and adaptively learn from sensor data, which may be reported back to the user without requiring expertise or training in sleep analysis and diagnosis, or further used to monitor sleep patterns and sleep quality over time.

By collecting individual sleep data, collecting user feedback, and learning from other users, or any combination thereof, the thermal system may have the added benefit of driving its thermal-comfort profile further towards a threshold determined as disruptive to sleeping. This may be particularly useful as tolerances and sensitivities to thermal stimulation may from person-to-person, season-to-season (or based on prevailing environmental conditions), or over time, for example, due to an acquired tolerance (or intolerance). In this way, the thermal system and method may be adaptive to the user and maintain its effectiveness, notwithstanding changed circumstances.

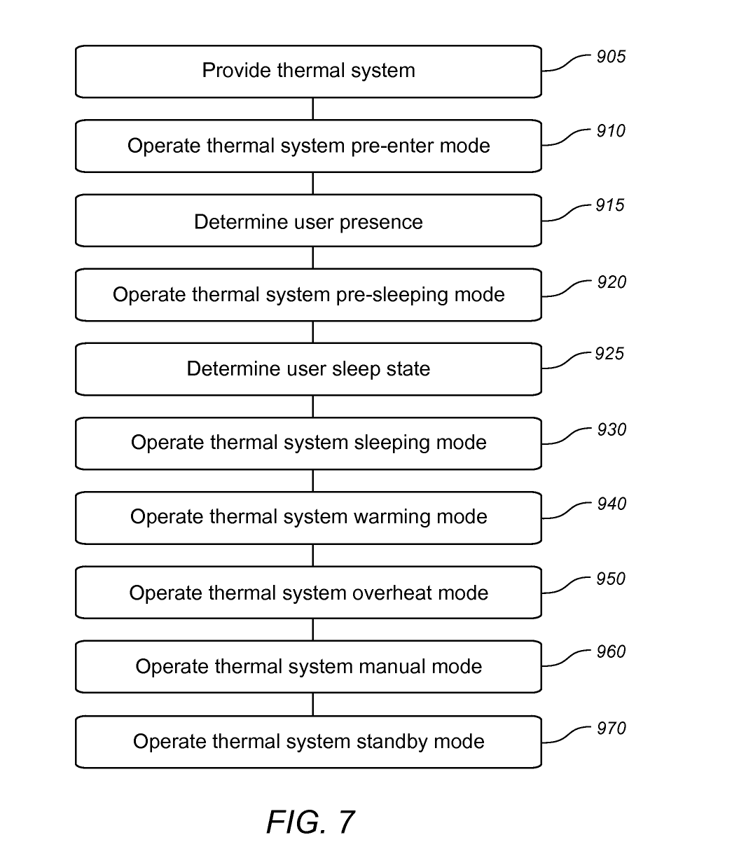

FIG. 7 is a flow chart of an exemplary method for conditioning a sleep environment. In particular, a sleep environment, such as a bed, may be conditioned for sleep using all or part of the thermal system 100 described above according to the foregoing method. Moreover, the skin temperature of a user may be managed during sleep such that sleep comfort and/or quality may be improved. The method may begin with step 905, providing a thermal system, as described above, including the heat exchanger 200 and the controller 500, or their variants or equivalents.

The method may include step 910, operating a pre-enter mode of the thermal system, as described above. Operating the pre-enter mode may include receiving an initiation command, and operating the heat exchanger to precondition the sleep environment as described above. As above, the initiation command may be provided manually by the user or automatically.

The method may include step 920, operating a pre-sleeping mode of the thermal system, as described above. Operating the pre-sleeping mode may include operating the heat exchanger to condition the sleep environment according to a thermal-comfort profile, as described above. According to one embodiment, the method may further include step 915, detecting the presence of the user, as described above. Upon detecting the presence of the user, the pre-sleeping mode may initiate in response. According to one embodiment, detecting the presence of the user may distinguishably include both detecting the presence of the user in contact with the user interface, and detecting the proximity of the user to the thermal system.

The method includes step 930, operating a sleeping mode of the thermal system, as described above. Operating the sleeping mode may include conductively cooling the user along a thermal-comfort profile to a minimum temperature. Conductively cooling the user along the thermal-comfort profile may include aggressively cooling the user as described above. According to one embodiment, the method may further include step 925, determining a sleep state of the user, as described above. Upon determining the sleep state of the user, the sleeping mode may initiate in response. Operating the sleeping mode may further include conductively warming the user or providing a thermal rest period (e.g., ceasing to cool the user or holding the measured temperature constant), intermittently. In addition, operating the sleeping mode may further include receiving and responding to a user input or automated data, so as to modify the operation of the heat exchanger.

The method also includes step 940, operating a warming mode of the thermal system, as described above. Operating the warming mode of the thermal system may include conductively warming the user toward a waking temperature at the waking time. Conductively warming the user along the thermal-comfort profile may include gently warming the user as described above. Operating the warming mode may further include conductively cooling the user or providing a thermal rest period (e.g., ceasing to cool the user or holding the measured temperature constant) intermittently. Operating the sleeping mode may further include receiving and responding to a user input or automated data, so as to modify the operation of the heat exchanger.

The method may include step 910, operating a pre-enter mode of the thermal system, as described above. Operating the pre-enter mode may include receiving an initiation command, and operating the heat exchanger to precondition the sleep environment as described above. As above, the initiation command may be provided manually by the user or automatically.

The method may include step 920, operating a pre-sleeping mode of the thermal system, as described above. Operating the pre-sleeping mode may include operating the heat exchanger to condition the sleep environment according to a thermal-comfort profile, as described above. According to one embodiment, the method may further include step 915, detecting the presence of the user, as described above. Upon detecting the presence of the user, the pre-sleeping mode may initiate in response. According to one embodiment, detecting the presence of the user may distinguishably include both detecting the presence of the user in contact with the user interface, and detecting the proximity of the user to the thermal system.

The method may further include step 950, operating an overheat mode of the thermal system, as described above. Operating the overheat mode may include operating the heat exchanger to heat the sleep environment to well above thermal comfort levels for a period of time, as described above. Operating the overheat mode may include heating the sleep environment to an overheat temperature between a begin-overheat time and an end-overheat time. The overheat temperature and the period of time may be selected for removing moisture, pasteurizing, sterilizing, or otherwise disinfecting the sleep environment.

The method may further include step 960, operating a manual mode of the thermal system, as described above. Operating the manual mode of the thermal system may include overriding a controller operation of the heat exchanger and/or the user directly operating the heat exchanger independently of a thermal-comfort profile.

The method may include step 970, operating a standby mode of the thermal system. Operating the standby mode may generally include powering down high energy components, such as the heat exchanger, while continuing to operate low power components, such as sensors, learning algorithms, and/or low power lighting. According to one embodiment, operating the standby mode may occur during all time outside of the operation modes described above. According to another embodiment, operating the standby mode may occur during a subset of all outside of the operation modes described above, such as during evening hours, when a user is detected proximate the thermal system, or when the thermal system has been remotely activated (such as upon registering as a hotel guest), to name a few.

The preceding detailed description is merely exemplary in nature and is not intended to limit the invention or the application and uses of the invention. The described embodiments are not limited to use in conjunction with a particular type of sleep environment or user application. Hence, although the present embodiments are, for convenience of explanation, depicted and described as being implemented in a thermal system pad placed on a bed, it will be appreciated that it can be implemented in various other types of sleep environments, and in various other furniture platforms and applications. Furthermore, there is no intention to be bound by any theory presented in any preceding section. It is also understood that the illustrations may include exaggerated dimensions and graphical representation to better illustrate the referenced items shown, and are not consider limiting unless expressly stated as such.

1. A system for thermally conditioning a sleep environment, the system comprising:
a heat exchanger configured to conductively heat and cool a user; and
a controller configured to operate the heat exchanger according to a sleeping mode and a warming mode,
the sleeping mode occurring between a begin-sleeping time and a begin-warming time, the sleeping mode including conductively cooling the user along a thermal-comfort profile to a minimum temperature, the thermal-comfort profile and the minimum temperature being proximate and above a threshold determined as disruptive to sleeping,
the warming mode occurring between the begin-warming time and a waking time, the warming mode including conductively warming the user toward a waking temperature at the waking time.

2. The system of claim 1, wherein the heat exchanger includes
a user interface configured to conductively heat and conductively cool the user via a fluid coolant in a thermal circuit, and
a heat module fluidly coupled to the user interface, the heat module, including a cooler configured to discharge heat from the fluid coolant, and a heater configured to supply heat to the fluid coolant.

3. The system of claim 1, wherein the thermal-comfort profile extends from a begin-sleeping temperature at the begin-sleeping time to the minimum temperature at the begin-warming time; and
wherein the minimum temperature is at least 5 degrees Celsius lower than the begin-sleeping temperature.

4. The system of claim 1, further comprising at least one user sensor configured to sense a presence of the user proximate the sleep environment and communicate said presence of the user to the controller; and,
wherein the controller is further configured to operate the heat exchanger according to a pre-sleeping mode, the pre-sleeping mode including at least one of conductively warming and conductively cooling the user prior to the begin-sleeping time, and in response to the presence of the user communicated from the at least one user sensor.

5. The system of claim 1, further comprising at least one environment sensor configured to sense at least one environmental condition proximate the sleep environment, and communicate said at least one environmental condition to the controller; and
wherein the controller is further configured to operate the heat exchanger according to a pre-sleeping mode, the pre-sleeping mode including at least one of conductively warming and conductively cooling the sleep environment prior to the begin-sleeping time, and in response to the at least one environmental condition communicated from the at least one environment sensor.

6. The system of claim 1, wherein the controller is further configured to operate the heat exchanger according to an overheat mode, the overheat mode occurring between a begin-overheat time and an end-overheat time, the overheat mode including heating the sleep environment to an overheat temperature of at least 40 degrees Celsius.

7. The system of claim 1, further comprising at least one user sensor configured to sense at least one metric of the user, and communicate said at least one metric of the user to the controller, the at least one metric of the user including at least one of a user's presence, temperature, temperature distribution, body position, body motion, heartbeat, breathing, muscle signaling, blood oxygenation, brainwave activity, skin conductance, and sleep state.

8. The system of claim 7, wherein the thermal-comfort profile a predetermined course of operation of the heat exchanger; and
wherein the controller is further configured to modify said predetermined course of operation of the heat exchanger in response to the at least one metric of the user communicated to the controller.

9. The system of claim 7, wherein the at least one user sensor includes a device worn by the user and operable when removed from the sleep environment.

10. The system of claim 7, wherein the at least one user sensor is further configured to sense at least one metric of another user, and the controller is further configured to distinguish between a plurality of users.

11. The system of claim 6, wherein the controller includes a processor, a memory, and a communication port, the controller being communicably coupled to the heat exchanger and the user sensor via the communication port; and
wherein the controller is further configured to collect, correlate, and adaptively learn from data communicated from the at least one user sensor.

12. A system for managing skin temperature of a user, the system comprising:
a user interface heat exchanger configured to conductively heat and cool a user;
a heat pump module fluidly coupled to the user interface heat exchanger, the heat pump module configured to conductively heat and conductively cool the user; and
a controller communicably coupled to the heat pump module, the controller configured to operate at least one of the heat pump module according to a sleeping mode and a warming mode,
the sleeping mode occurring between a begin-sleeping time and a begin-warming time, the sleeping mode including conductively cooling the user along a thermal-comfort profile to a minimum temperature, the thermal-comfort profile and the minimum temperature being proximate and above a threshold determined as disruptive to sleeping,
the warming mode occurring between the begin-warming time and a waking time, the warming mode including conductively warming the user toward a waking temperature at the waking time.

13. A method for thermally conditioning a sleep environment, the method comprising:
providing a thermal system including a heat exchanger, the heat exchanger configured to conductively heat and cool a user;
operating a sleeping mode of the thermal system, including conductively cooling the user along a thermal-comfort profile to a minimum temperature between a begin-sleeping time and a begin-warming time, the thermal-comfort profile and the minimum temperature being above a threshold thermal profile determined as disruptive to sleeping; and
operating a warming mode of the thermal system, including conductively warming the user toward a waking temperature between the begin-warming time and a waking time.

14. The method of claim 13, further comprising detecting a sleep state of the user; and
wherein the operating the sleeping mode of the thermal system is in response to the detecting the sleep state of the user.

15. The method of claim 13, wherein at least one of the operating the sleeping mode of the thermal system and the operating the warming mode of the thermal system further includes
receiving at least one of a user input or automated data, and
modifying operation of the heat exchanger in response to the user input or automated data.

16. The method of claim 13, wherein the operating the sleeping mode of the thermal system further includes conductively warming the user or providing a thermal rest period along a thermal-comfort profile intermittently; and
wherein the operating a warming mode of the thermal system further includes at least one of conductively cooling the user and providing a thermal rest period along a thermal-comfort profile, intermittently.

17. The method of claim 13, further comprising operating a pre-enter mode of the thermal system, including receiving an initiation command, and operating the heat exchanger to precondition the sleep environment to a predetermined temperature.

18. The method of claim 13, further comprising operating a pre-sleeping mode of the thermal system, including
detecting the presence of the user,
operating the heat exchanger to condition the sleep environment according to a thermal-comfort profile,
receiving at least one of a user input or automated data, and
modifying operation of the heat exchanger in response to at least one of the user input or automated data.

19. The method of claim 13, further comprising operating an overheat mode of the thermal system, including operating the heat exchanger to heat the sleep environment to at least 40 degrees Celsius.

20. The method of claim 13, further comprising operating a manual mode of the thermal system, including at least one of overriding a controller operation of the heat exchanger and the user directly operating the heat exchanger independently of a thermal-comfort profile.
